(19) United States
(12) Patent Application Publication
(10) Pub. No.: US 2020/0121545 A1
(43) Pub. Date: Apr. 23, 2020

(54) Intermittent Pneumatic Compression System

(71) Applicant: Peachtree Medical Products, LLC, Charleston, SC (US)

(72) Inventors: Richard L. Parker, SR., Folly Beach, SC (US); Sarah R. Buck, Snellville, GA (US); Dershika Patel, Stone Mountain, GA (US); Michael A. Fisher, Grayson, GA (US); Nicholas L. Hilborn, Morrisville, NC (US); Daniel Bonistalli, Atlanta, GA (US); Patrick Strane, Atlanta, GA (US)

(21) Appl. No.: 16/655,987

(22) Filed: Oct. 17, 2019

Related U.S. Application Data
(60) Provisional application No. 62/746,799, filed on Oct. 17, 2018.

(51) Int. Cl. A61H 9/00

(52) U.S. Cl. CPC A61H 9/0078; A61H 2201/164; A61H 2201/165; G16H 10/65; A61H 2201/0157; A61H 2201/501; A61H 2201/5058

(57) ABSTRACT

The invention is directed at an intermittent pneumatic compression (IPC) system that is modular in nature and allows mobility for subjects utilizing the system. In an aspect, the IPC system utilizes a mobile IPC device. The mobile IPC devices is an easy to use portable device that can be mounted onto a limb of a subject. The modular mobile IPC device includes an independent power source, allowing subjects to be mobile while the IPC device is operating. In an aspect, the mobile IPC device also ensures compliance of following the prescribed treatment.

### REFERENCE TO RELATED APPLICATION

This application claims priority to U.S. Provisional Application No. 62/746,799, filed on Oct. 17, 2018, which is relied upon and incorporated in its entirety by reference.

### FIELD OF INVENTION

This invention generally relates to an intermittent pneumatic compression device for use with limbs to improve venous circulation.

### BACKGROUND OF THE INVENTION

Intermittent pneumatic compression (IPC) devices are used to improve venous circulation in limbs. In most aspects, IPC devices are used to improve venous circulation in the legs of patient suffering edema, or patients who may be at risk for developing blood clots in the deep veins of the leg, typically due to decreased blood flow and a higher likelihood of clotting due to low blood flow. IPC devices intermittently provide high pressure to the tissues of the limb, forcing fluids such as blood and lymph out of the pressurized area. Following the application of high pressure, pressure is reduced allowing blood to flow back into the limb.

While current IPCs help prevent blood clots and improve circulation within limbs suffering edema, these IPCs still have several drawbacks, especially failing to adequately satisfying clinical needs. Many IPC devices are in fact stationary IPC devices. Some IPC devices do not themselves provide pneumatic pressure—they rely on external pneumatic controllers that are connected via a tube to a compression sleeve that provides the pressure to the tissue of the limb of the subject. The design and operation of products that communicate pressure to the sleeve using external tubing requires patients to be attached to an external intermittent pneumatic compression device, which are immobile themselves. This design does present significant inconvenience and risk to both the patient and the hospital staff due to entanglements and the need to manage external cables and conduits. Despite this, hospitals tend to use these devices poses less risk than devices that tether patients to electrical power sources.

Current products use electrical energy to create therapeutic super-atmospheric pneumatic pressure that is applied to the patient. However, many IPC devices do not have their own power sources, and must be connected to electrical outlets. Current products use standard 120 Volt 50/60 Hz AC wall power to operate. These non-mobile versions of IPCs are too bulky, and restrict the movement of the subject to a limited area, or in many cases, a stationary state.

Current mobile IPC devices that tubeless and have their own power sources (e.g., battery powered) provide too weak a pressure to ultimately be effectively, or fail to hold their charge for sufficient lengths of time to apply the needed therapy. The former have inferior clinical outcomes, and the latter cause excessive work for nurse resources who must remove, recharge, and re-apply the IPC device. In addition, most have to be plugged into a wall to be charged. In either case, currently available mobile IPC devices cannot be used continuously for a period of 18 hours in a day efficiently to meet the desired results as planned by physicians prescribing such course of treatment without being recharged while in use, requiring the user to be immobile. In addition, the current IPC devices, mobile and immobile, present the risk of electrocution and other electrical fault conditions because of the need to be connected to wall power for at least time during operation of the IPC device. For mobile patients, being tethered by an electrical cable to wall power is at least inconvenient and defeats the intent of a mobile device. For bedridden patients, having a power source attached to the patient while in the bed and under covers exposes the patient to various electrical risks (electrocution, ground, user entanglement, trip hazards, wire breakage, accidental disconnection, confusion with other lines in the field and other phenomena). Because of these risks, hospitals currently do not use mobile IPC devices where power cables are attached at any point to the patient.

Further, it is important that physicians can make sure that patients are complying with the prescribed treatment plan. In such instances, the IPCs are tethered to a computer in order to ensure that the patient is maintaining the treatment plan, severely impacting the quality of life of the patient.

In addition, many portable IPC devices are only configured to use with a single patient. That is, once an IPC device is been used by a patient, for sanitary purposes, it cannot be used by additional users. These devices are completely disposable and do not enable cleaning or recycling of the components. The single-use nature of these mobile IPC devise requires the use of materials and components that are sourced for low cost and not for reliability or performance. These lower-quality devices tend to have few features, cheap sleeves, and inexpensive controllers that result in variable performance that risk good patient outcomes.

In some mobile IPC devices, controllers can be removably attached to the sleeve, the pneumatic controller is either carried by the patient and is tethered to the sleeve by a pressure conduit or the pneumatic controller is removably attached directly to the sleeve. In such instances, the pneumatic controller can be removed from the sleeve, but requires detaching a pneumatic tether from the controller. The dissembled devise consists of a pneumatic controller and a sleeve with a very long pneumatic conduit hanging off to the side, which is difficult to manage and is a source of irritation to the user.

The mobile pneumatic controllers that are removably attachable to the sleeve use two methods of attachment—hook and loop fasteners and mechanical clasps. The hook and loop fasteners create a mechanical connection between the hook-receptive sleeve and hooks on the bottom surface of the pneumatic controller. These hook and loop portable IPC devices typically have a loose and weak attachment between the pneumatic controller and the sleeve. Such weak connections lead to attachment fails in several mechanical conditions that occur during normal use like vigorous leg movement or when the IPC device bumps into objects during ambulation. Another drawback is that these fails over time lead to the hooks becoming broken or clogged with debris and/or the hook-receptive surface frays and loses the ability to attach to the hooks on the pneumatic controller. The other IPC devices that use mechanical clasps to removably attach the controller to the sleeve suffer from the inconvenience of having to undue multiple mechanical attachments before the pneumatic controller can be removed from the sleeve. The mechanical claps used for some of these devices do not appear to be robust. Overall, the designs used for portable IPC devices allow for too much user error and lead to user frustration.

Most IPC devices utilize a pneumatic bladder to deliver mechanical energy into the underlying tissues to promote circulation within those tissues. In these devices, the bladder is usually contained within a sleeve that is used to attach the bladder to the patient. The sleeve and bladder combinations of the prior art have their drawbacks. The sleeves usually interface with the skin of a patient. Therefore, the sleeves are exposed to heat and moisture from the subject's skin. If the sleeve is not properly constructed to conduct heat and moisture away from the skin, the sleeve can cause the patient great discomfort. If not properly constructed, skin irritations, caused by trapped heat and moisture, can result. Along the same lines, some sleeves have been constructed with extensions or mounts that dig into the skin of the patient, as well as being made out of materials that have a bad chemical reaction with the skin of a patient.

Prior art sleeves have also failed to conform to the complex user anatomy while maintaining sufficient mechanical integrity to hold the bladder in place during use and convey mechanical energy into the underlying tissues. If the sleeve material is too compliant (flexible), the sleeve will stretch when the bladder inflates and the mechanical energy intended for tissue therapy will be made less effective by stretching the fabric instead of compressing the underlying tissues. If the sleeve material is too stiff (rigid), the sleeve will not be able to wrap and accommodate the underlying tissue intending to be treated. In this instance, the sleeve will wrinkle when applied to the complex shape of the user's tissue anatomy. The wrinkled sleeve may enable the pneumatic bladder to migrate within the sleeve when inflated or the bladder could inflate within the wrinkled space and apply energy to the rigid sleeve material instead of the underlying tissues, thus reducing the user's therapeutic experience.

The pneumatic bladders used in intermittent pressure cuff devices (IPCD's) must be contained within the pressure cuff and oriented to the patient's anatomy during use, which can exceed 18 hours in a 24-hour period. The bladder imparts the therapeutic mechanical force into the patient's tissues. A sleeve/cuff holds the bladder in the proper location to deliver the therapy. If the bladder is improperly located or allowed to migrate, the patient will not benefit from the IPCD therapy. Most mechanical cuffs have a pocket to locate the bladder. In some instances, the bladder is fixed inside a form-fitting pocket that enables insertion of the bladder. In other instances, the bladders contain regions within the pressurized area where material has been removed so the cuff material can be sealed within the region to create a spot weld that holds the bladder in place during placement and inflation. In other instances, the bladders contain regions within the pressurized area that enable stitching or piercing of the bladder material to affix the bladder within the cuff. To maintain the pneumatic integrity of the bladder, the regions containing the spot welds and/or stitches must have a perimeter seal. Adding seals constrains the ability of the bladder to inflate to a larger size since the edges are welded together. Adding new weld seals also increases the risk of pneumatic leaks. Another liability of these seals within the bladder's inflated region is the increase in geometric complexity and the likelihood of introducing stress raisers in the inflated envelope that can lead to either burst failures or fatigue failures (cyclic inflation causes mechanical damage due to the presence of a weld that accumulates over time).

A typical pneumatic bladder has a continuous and smooth peripheral weld that defines a region inside the bladder that is the active inflation region. Tubing transcends the peripheral weld (a mandrel weld region) to inflate the bladder. Alternatively, surface mount connectors are used to inflate the bladder and do not interfere with the peripheral weld (not shown). No salvage edge is shown as it is either trimmed or manufactured to be flush with the peripheral weld.

In addition, most sleeve/bladder combinations are done so that the bladder and sleeve are permanently attached to one another. That is, there is no way to remove the bladder from the sleeve and/or the other components of the IPC device, so that when the sleeve fails, the bladder is discarded with the sleeve.

Therefore, there is a need for an IPC device that can provide adequate power and battery life, while being modular such that a nurse can recharge the battery without needing to remove and reapply the sleeve. In addition, there is a need for an IPC device that is reusable while hygienic. Further, there is need for an IPC device that can also ensure that compliance occurs with the prescribed treatment plan. There is also a need for an IPC device that is easy to maintain and is reliable in function.

There is also a need for a sleeve that has a chemical and physical composition of matter that does not irritate an individual's skin. In addition, there is a need for a sleeve that has high breathability, allowing the sleeve to conduct heat and enable the skin to communicate moisture away from the skin-sleeve interface. There is also a need for the sleeve to have sufficient mechanical properties to enable delivery of the therapeutic energy from the bladder to the underlying tissues, maintain the position of the bladder on the targeted region of the patent while also enabling the pressure-delivery system to attach to the bladder. In addition, there is a need for a sleeve that is elastic enough to conform to the anatomy of the patient when in use but rigid enough to ensure that the mechanical energy of the pneumatic bladder is applied to the targeted tissue, and not aware from the tissue. There is also a need for a device with better bladder construction, as well as bladder construction that can be reused when the sleeve has failed.

### SUMMARY OF THE INVENTION

This invention relates to an intermittent pneumatic compression (IPC) system that is modular in nature and allows mobility for subjects utilizing the system in an aspect, the IPC system utilizes a mobile IPC device. In an aspect, the mobile IPC device is configured to provide sufficient pressure to extremities to satisfy the need of treatment. The mobile IPC device is an easy to use portable device that can be mounted onto a limb of a subject. The mobile IPC device is prescribed by a physician, and can be used for inpatients and outpatients to help prevent the onset of deep vein thrombosis by stimulating blood flow in the extremities. In such aspects, the IPC device is configured to stimulate blood flow through simulating muscle contractions. The modular mobile IPC device includes an independent power source, allowing subjects to be mobile while the IPC device is operating. In an aspect, the IPC system utilizes a plurality of mobile IPC devices that can be deployed simultaneously to numerous patients as needed.

In an aspect, the mobile IPC device of the IPC system includes a driving component that controls the inflation and deflation of an inflatable sleeve that engages a limb of the subject. The driving component can include housing that contains a pump subsystem, a computing device, and a self-contained power source. The driving component can be removably attached to the inflatable sleeve. The combination of the driving component and the inflatable sleeve of the mobile IPC device allows mobility for the subject. In an aspect, the pump subsystem of the driving component is controlled in operation by the computing device to inflate and deflate the inflatable sleeve. In an aspect, the pump subsystem utilizes a pneumatic pump. The pneumatic pump utilizes air as a fluid to transmit mechanical energy through the inflatable sleeve, which applies the compression to the targeted area of the limb of the subject.

In an aspect, the computing device of the driving component is configured to control the cycle time, display, and pump subsystem. In another aspect, the computing device can include a plurality of sensors that track the activity of the pump subsystem, the status of the self-contained power source, as well as the activity of the subject. In an aspect, the computing device can also include a user interface and/or display that shows the status of treatment (inflation, deflation, how many hours treatment has been applied, etc.). The computing device can also include communication means, allowing the computing device to communicate back the activity of the subject and operation of the IPC device to a remote server or remote computing device to ensure compliance with the prescribed treatment. In another aspect, the mobile IPC device can include several other sensing modalities to aid and refine the monitoring of patient health.

In aspect, the self-contained power source of the mobile IPC device can include a battery. In such aspects, the battery is configured to be rechargeable or easily replaced. In aspects in which the mobile IPC device contains a rechargeable battery, the IPC system can utilize charging stations that can recharge several mobile IPC devices at once. Such a docking/recharging station can be utilized in a hospital or clinic setting, allowing multiple mobile IPC devices to be charging while having others being used by patients.

In another aspect, the mobile IPC device is configured to ensure that the patient is complying with the prescribed treatment schedule, through tracking of actual use of the IPC device over a given time period. For example, the mobile IPC device is configured to keep track of the time the subject wears the mobile IPC device and the application of the prescribed treatment to the subject through the mobile IPC device, as well as how many days the subject has complied with the daily treatment regimen.

In another aspect, the mobile IPC device is configured to work with an inflatable compression sleeve that is placed on a limb of the subject. In an exemplary aspect, the mobile IPC device is configured to be removably connected to the inflatable compression sleeve. In such aspects, the inflatable compression sleeve can assigned to an individual subject, allowing the mobile IPC device to be used by multiple subjects in a hygienic manner.

In an aspect, the pump subsystem can be configured to provide high, intermittent pressure in a regular time frequency. Further, the pump power supply can be battery-powered and can supply power for a suitable clinically recommended daily duration (e.g., 18 to 24 hours per day) of a period of recommended days (e.g., 10-90 days). In an aspect, the power supply is modular and can be disconnected from the sleeve for ease in recharging.

In an aspect, the majority of the functioning components of the IPC power (e.g., power supply, pump, hardware, and firmware) are configured to be removable from the compression sleeve. In an exemplary aspect, the bladder can also be configured to be removable from the compression sleeve. In such aspects, the compression sleeve can be disposable, while being able to retain the remaining components of the IPC device for additional uses, saving costs, and reducing the waste stream associated with the use of the system.

In another aspect, the body of the sleeve is comprised of an outer textile that is selected to enhance patient comfort. In such aspects, a portion or the entire portion of the textile configured for contact with the tissue of the patient is lined with a high-durometer material and/or a high co-efficient of friction which prevents the sleeve from migrating on the limb of the patient. In such aspects, the IPC device is smaller, more mobile solution compared to current electrical outlet powered IPC devices, while still providing adequate power, adequate battery life, a bladder in some cases, and hygienic use between several subjects while done so in a costly fashion.

In an aspect, the power supply is configured to be removable from the IPC device. The removable power supply can be enabled as a battery pack. As the power supply is spent operating the IPC, the removable battery pack is removed and replaced by a battery pack with a fresh charge. The removable battery pack can be rechargeable. The IPC device is configured to only be operable by the removable battery packs and no wall-power or charger can be used to operate the IPC device. This design configuration prevents users from risks associated with being tethered to wall power or other power supplies. This design feature also enables the IPC to be used in various operational environments that typically challenge battery operated devices because a cold/hot or failing removable power supply can be replaced by a new removable power supply to ensure continuous operation of the IPC despite the challenging environment.

In an aspect, the mobile IPC device is configured to aid in the prevention of DVT, enhance blood circulation, diminish post-operative pain and swelling, and reduce wound healing time. In an aspect, the mobile IPC device is configured to aid in the treatment and healing of stasis dermatitis, venous stasis ulcers, arterial and diabetic leg ulcers, chronic venous insufficiency, and reduction of edema in the lower limbs.

In an aspect, the mobile IPC device utilizes a pneumatic bladder with a continuous and smooth peripheral weld that defines a region inside the bladder that is the active inflation region.

In an aspect, the invention is directed at a mobile intermittent pneumatic compression (IPC) device including a driving component that is removably mounted to an inflatable sleeve. In such aspects, the driving component includes a removable power source, a pump subsystem, a computing device configured to co the pump subsystem; and, housing containing the self-contained power source, the pump subsystem, and the computing device. The pump system can inflate the inflatable sleeve when placed on a subject. In an aspect, the driving component is configured to be operable only with the removable power source is connected to the driving component. In another aspect, the removable power source is only rechargeable when the removable power source is disconnected from the driving component.

In some embodiments, the inflatable sleeve includes a sleeve, an inflatable bladder, and a mounting means to mount the driving component to the sleeve. The inflatable sleeve can include a unique identifying means so that the inflatable sleeve can be assigned to a specific patient. The unique identifying means can include an RFID chip. The sleeve of the inflatable sleeve can include a composite material system. The composite material system can include a stiff fabric and a flexible fabric. In an aspect, the inflatable bladder of the inflatable sleeve includes a salvage edge used for securing the inflatable bladder within the sleeve. In some instances, the inflatable sleeve is configured to be disposable. In such instances, it is possible for the inflatable bladder to be configured to be removed from the sleeve for reuse.

In an aspect, the invention is directed at a mobile IPC device that includes a driving component with a removable battery, a pump subsystem, a computing device configured to control the pump subsystem, and housing to contain those components, as a well as an inflatable bladder that is configured to be inserted into an inflatable sleeve. In such instances, the driving component is configured to be removably inserted and mounted to the inflatable sleeve. In some instances, the driving component is configured to be inoperable when the self-contained removable power source is removed and the self-contained removable power source is configured to be recharged only when removed from the driving component. The computing device can include a compliance meter that reports user compliance for a current 24-hour period and over a sequence of 24-hour periods. In addition, the computing device can be configured to be reset between use sessions or between users. In some instances, the computing device is configured to not store any patient-specific data nor is configurable by the patient. In some instances, the computing device is configured to apply a pre-configured pressure cycle.

Other features and advantages of the invention will become apparent to one with skill in the art upon examination of the following drawings and detailed description. It is intended that all such additional features and advantages be included herein within the scope of the present invention.

### BRIEF DESCRIPTION OF THE FIGURES

FIGS. 1-2 illustrate sleeve and bladder combinations known in the prior art.

FIG. 3 illustrates the construction of a bladder known in the prior art.

FIGS. 4-5 are top perspective views of a mobile intermittent pneumatic compression (IPC) device according to an aspect of the present invention.

FIG. 6 is an exploded view of components of the IPC device of FIGS. 4-5.

FIG. 7 is a front plan view of a driving component and sleeve mount of the IPC device according to an aspect of the present invention.

FIG. 8 is a side plan view of the driving component and sleeve mount of FIG. 7.

FIG. 9 is a top plan view of the driving component and sleeve mount of FIG. 7.

FIG. 9 is a bottom plan view of the driving component and sleeve mount of FIG. 7.

FIG. 11 is a perspective view of the driving component of FIG. 7.

FIGS. 11-15 are various views of the driving component of FIG. 7.

FIGS. 16-18 are various see-through views of the driving component of FIG. 7.

FIG. 19 is a exploded view of the driving component of FIG.

FIGS. 20-21 are perspective views of the housing of the driving component of FIG. 7 according to an aspect of the present invention.

FIGS. 22-24 are various perspective views of a removable battery of the driving component of FIG. 7 according to an aspect of the present invention.

FIG. 25 is a front plan view of a sleeve mount and sleeve of an IPC device according to an aspect of the present invention.

FIGS. 26 and 27 are various perspective views of the sleeve dock of FIG. 25 according to an aspect of the present invention.

FIGS. 28-29 are various plan views of the sleeve dock of FIG. 25.

FIG. 30 is a cross-sectional view of the sleeve dock of FIG. 29 along line B-B.

FIG. 31 is a perspective top view of an air flow port according to an aspect of the present invention.

FIG. 32 is a bottom perspective view of the air flow port of FIG. 31.

FIG. 33 is a cross-sectional view of the air flow port of FIG. 31.

FIG. 34 is an exploded view of components of a sleeve of an IPC device according to an aspect of the present invention.

FIG. 35 is atop perspective view of a bladder of FIG. 34.

FIG. 36 is a top perspective view of a port of the bladder of FIG. 35.

FIG. 37 is a top plan view of a sleeve and mount according to an aspect of the present invention.

FIG. 38 is a front plan view of a sleeve according to an aspect of the present invention

FIG. 39 is a top plan view of a battery charger according to an aspect of the present invention.

FIGS. 40-42 are various perspective views of battery chargers according to aspects of the present invention.

FIGS. 43-49 are schematic representations of various bladder formations according to aspects of the present invention.

FIGS. 50A-E are schematic representations of combinations of various bladders with sleeves according to aspects of the present invention.

FIG. 51 is a schematic representative of a sleeve and bladder on a subject according to an aspect of the present invention.

FIG. 52 is a schematic representation of a sleeve and bladder combination according to an aspect of the present invention.

FIGS. 53-58 are schematic representations of various bladder, driving component, and sleeve combinations according to aspects of the present invention.

FIGS. 59A-62B are schematic representations of various bladder constructions according to aspects of the present invention.

FIGS. 63-65 illustrate an IPC device according to an aspect of the present invention.

FIGS. 66-69 illustrate a mounting means for a driving component of the IPC device of FIGS. 63-65.

FIGS. 70-73, 73B, 74, and 74B illustrate a housing of the driving component of the IPC device of FIGS. 63-65.

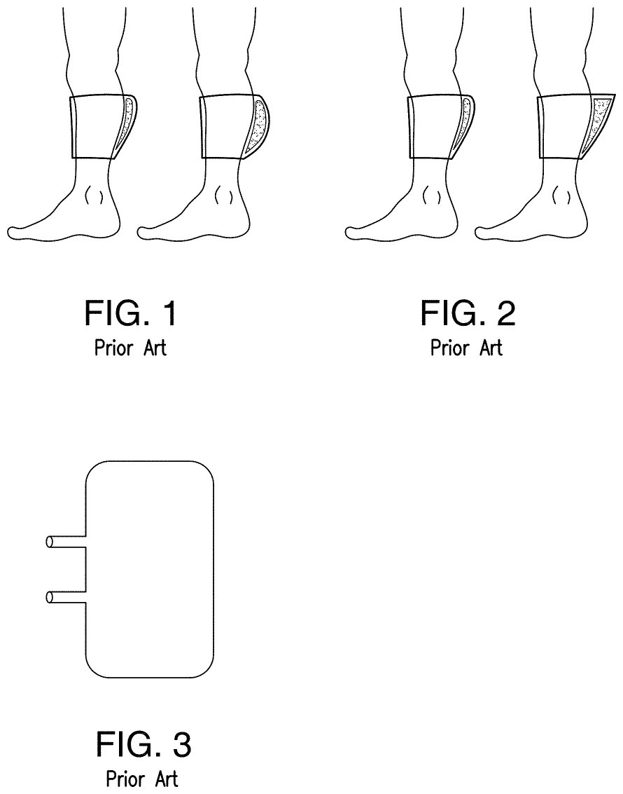

### DETAILED DESCRIPTION OF THE INVENTION

Embodiments of the present invention will be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

In the following description, numerous specific details are set forth. However, it is to be understood that embodiments of the invention may be practiced without these specific details. In other instances, well-known methods, structures and techniques have been shown in detail in order not to obscure an understanding of this description.

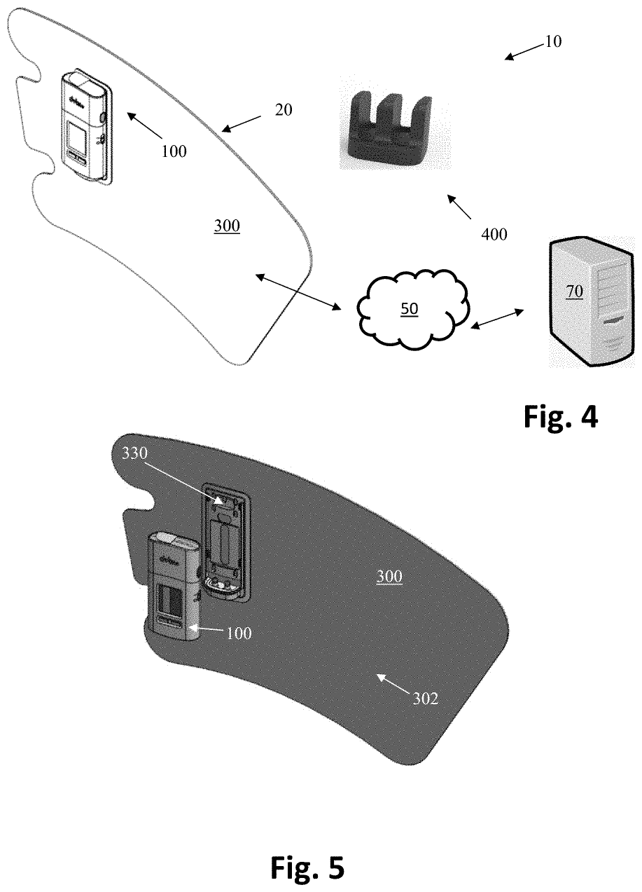

The present invention is directed towards a mobile intermittent pneumatic compression (IPC) device 20, as shown in FIG. 4, for use in an IPC system 10. The IPC device 20 is configured to communicate with a server 70 via wired communications or wireless communications over a network 50. In addition, the IPC system 10 includes a charging port for a power source of the IPC device 20, discussed in detail below.

In an aspect, the mobile IPC device 20 includes a driving component 100 and an inflatable sleeve 300. In one embodiment, the physical configuration of the IPC device 20 promotes ease of use for low dexterity patients by orienting the sleeve 300 and the driving component 100 such that their proper coupling to one another is facilitated, discussed in detail below. In an aspect, the mobile IPC device 20 is controlled by the driving component 100, which is configured to be removably attached to the inflatable sleeve 300. The inflatable sleeve 300 is configured to be secured on a limb of a subject, and applies the compression to the limb as directed by the driving component 100, both of which are discussed in detail below.

The American College of Chest Physician guidelines recommend that IPC devices should be used by patients undergoing major orthopedic surgery, especially portable, battery powered IPC devices capable of recording and reporting proper wear time on a daily basis for inpatients and outpatients. Efforts should be made to achieve 18 hours of daily compliance. The IPC device 20 of the present invention meets these recommendations, and can be utilized by inpatients and outpatients.

In an aspect, as shown in FIGS. 4-7, the mobile IPC device 20 is configured to be operated by a user without limiting the mobility of the user. That is, the mobile IPC device 20 in use is not tethered to any external component. There are no external cables (electrical or data connection) nor tubes (pneumatic) necessary to get the full use of the mobile IPC device 20. In an aspect, the mobility of the IPC device 20 is a direct result of the device including a removable self-contained power source 180 for the driving component 100, various levels of sophistication of information displays, and wireless communication means, all discussed in more detail below.

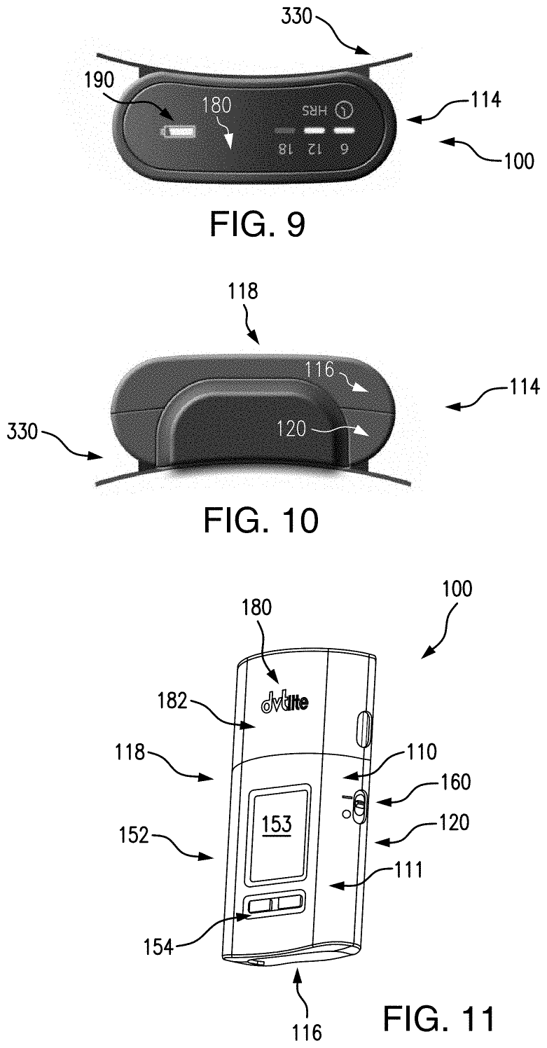

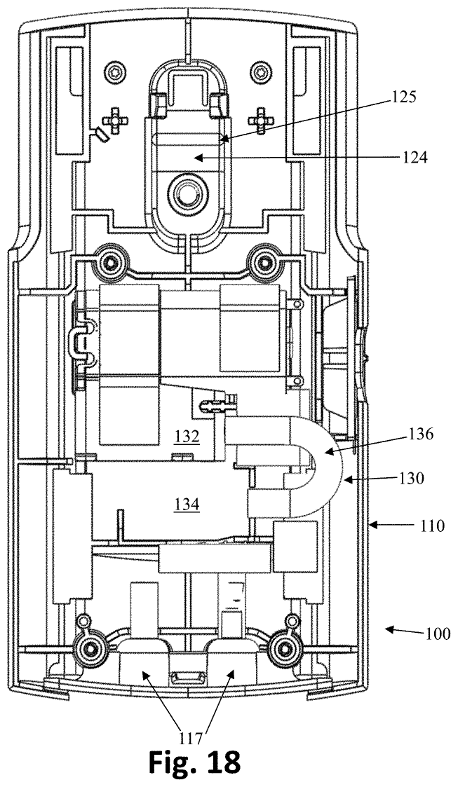

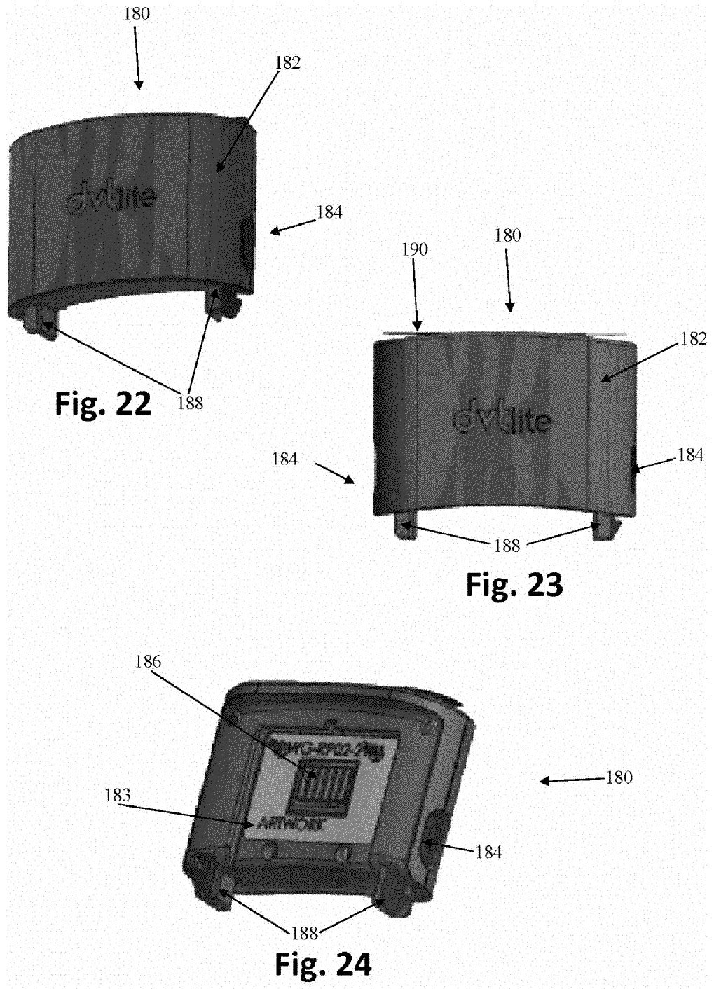

The driving component 100 contains the majority of the working components of the mobile IPC device 20 for the IPC system. In an aspect, as shown in FIGS. 4-24, the driving component 100 has a housing 110 that contains a pump subsystem 130, a computing device 150, and a self-contained power source 180. The self-contained power source 180 provides the power to the pump subsystem 130 and the computing device 150, wherein the computing device 150 is configured to control the pump subsystem 130 and operation of the IPC device 20.

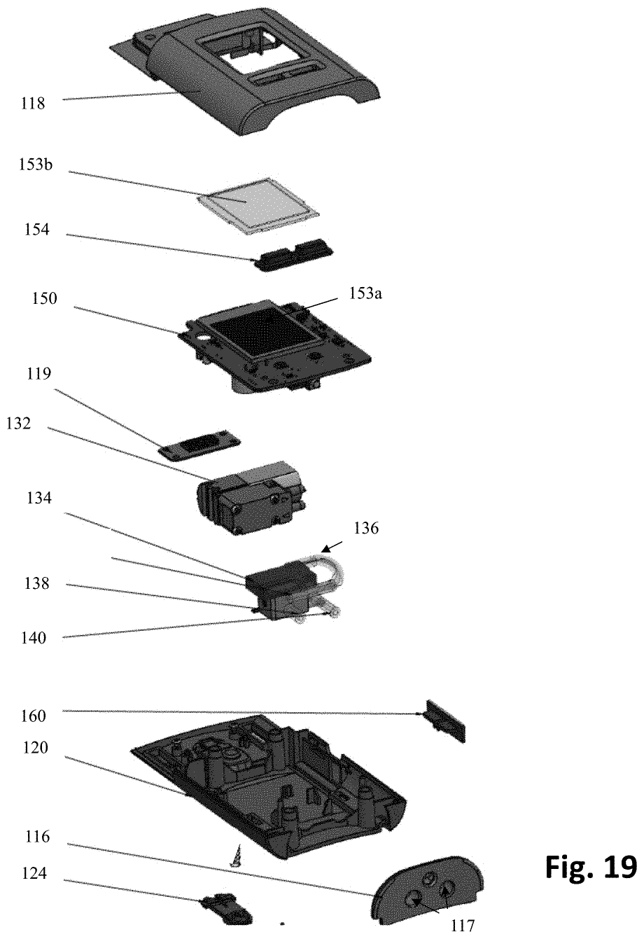

In an aspect, the housing 110 of the driving component 100 is made of a sufficiently hard material, including, but not limited to, ABS, polycarbonate, ASA, semi-rigid polyisoprene, hard rubber, and other plastic materials that are rugged and can be sterilized without becoming compromised. In another aspect, the housing can be made of machined metal alloys. In an aspect, the housing 110 is configured to contain or engage with all of the parts of the driving component 100. In an aspect, the housing can include a body 111 with a first end 114, a second end 116, a top portion 118 and a bottom portion 120 (when the driving component 100 is oriented in a horizontal position). As shown in FIG. 19, the top portion 118 and the bottom portion 120 can be two separate pieces that can be attached to one another. The second end 116 can be configured to have fluid communication port(s) 117 connected to the pump subsystem 130, and further configured to connect to the inflatable sleeve 300, discussed in detail below.

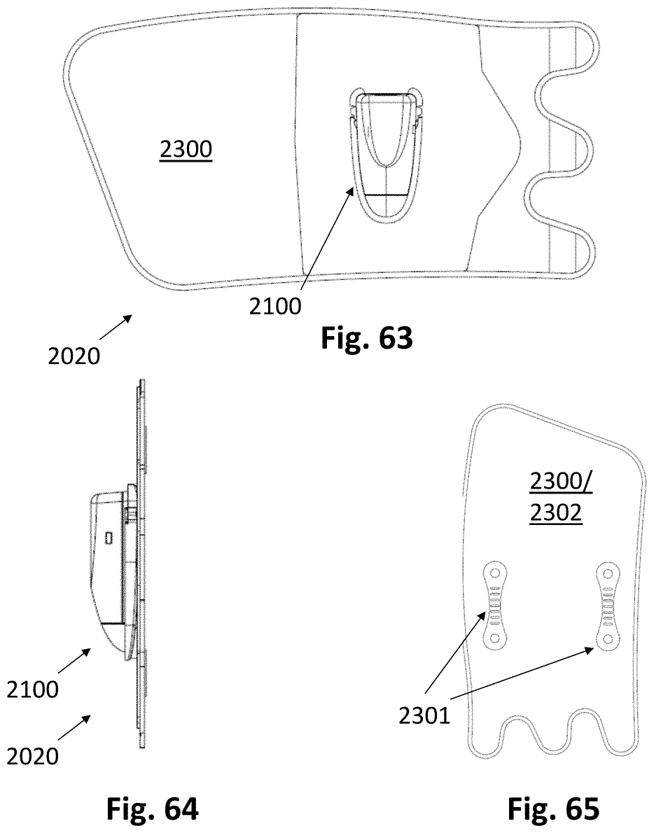

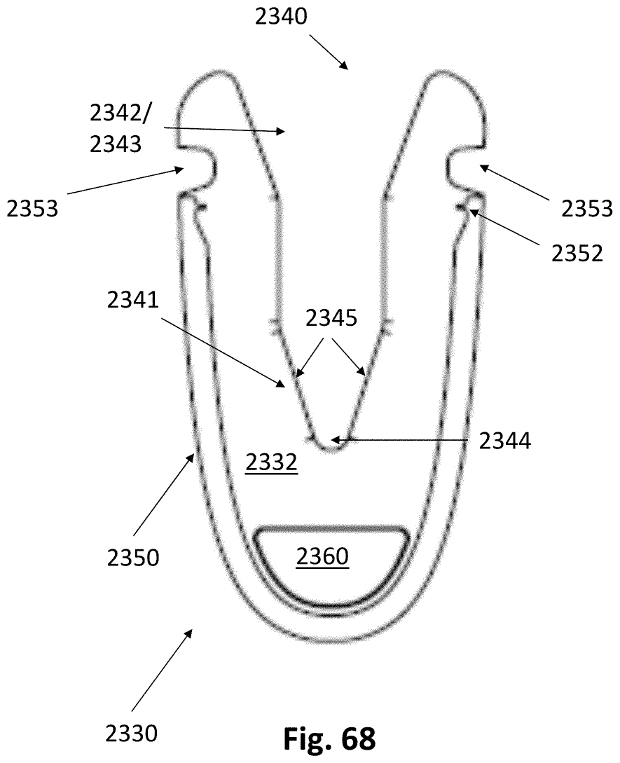

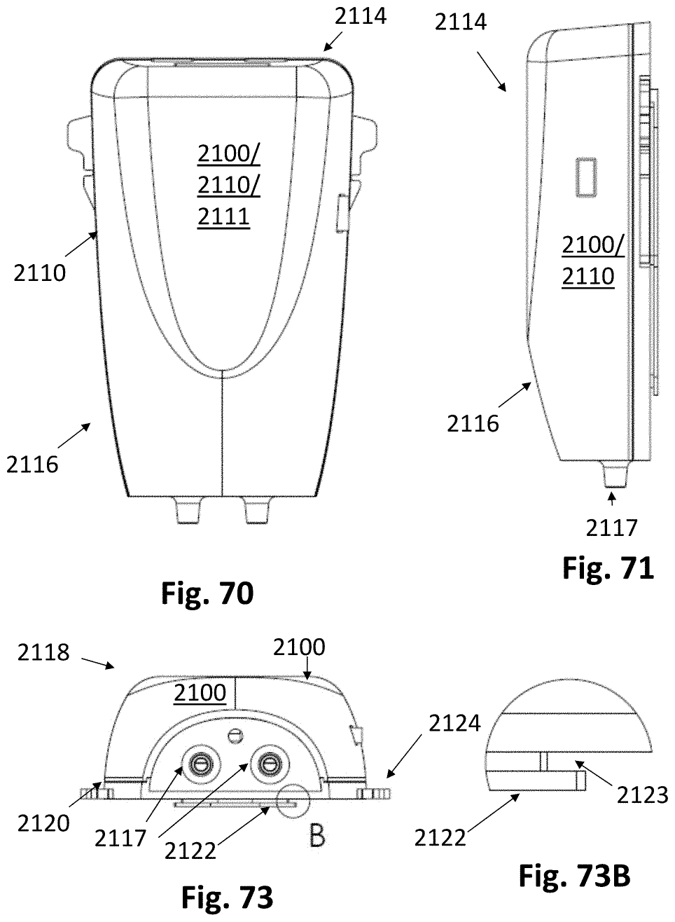

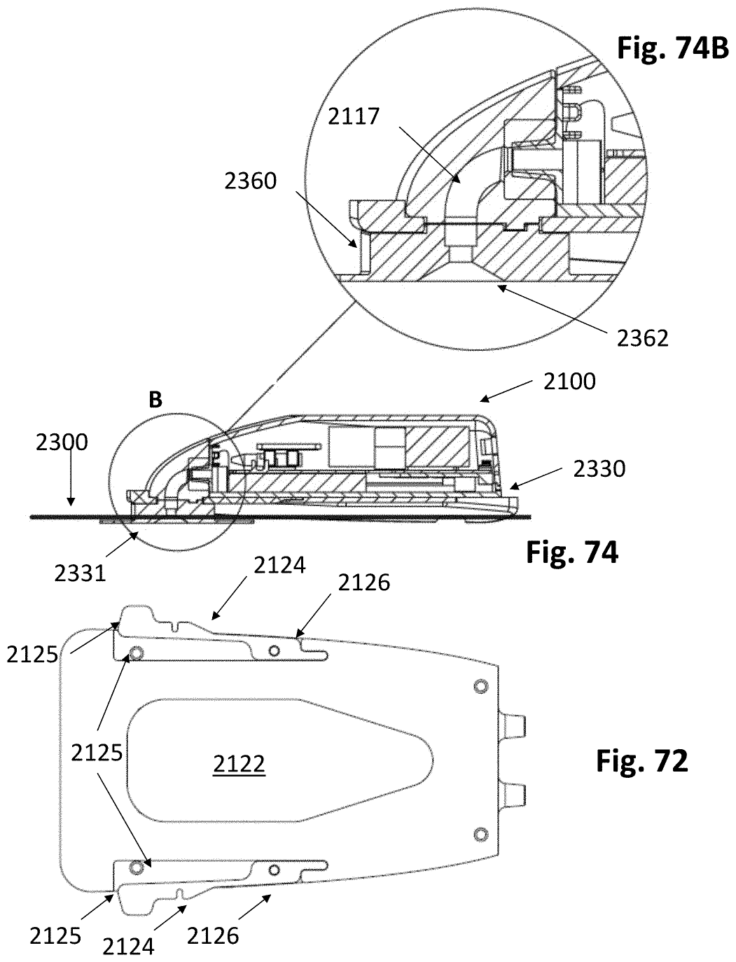

In an aspect, the housing 110 of the driving component 100 is rounded at the edges of the top portion 118 and bottom portion 120, which lessens the chance of a sharp edge getting caught on another surface, which could lead to damage as well as the driving component 100 being forced off the sleeve 300 if enough force is applied. In an aspect, the housing 110 has a width and height that is substantially the same throughout its length. In other embodiments, the housing 110 can include a tapered body, as discussed in more detail below in relation to FIGS. 63-75.

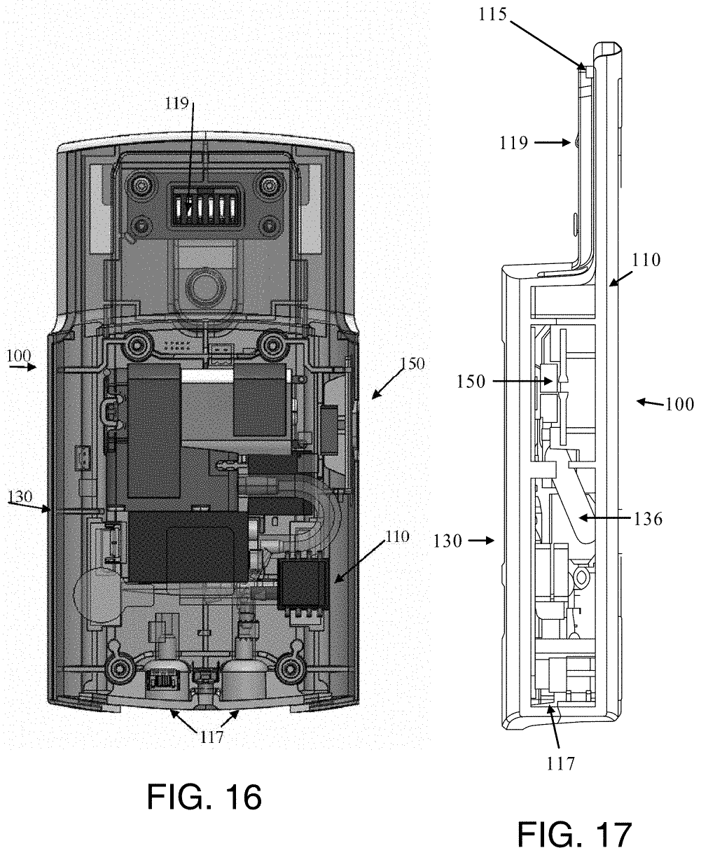

In an aspect, the bottom portion 120 of the body 111 of the housing 110 is configured to provide a base mount for the internal components of the driving component 100 (see FIGS. 16-19). Further, the top portion 118 can be configured to be removable from the bottom portion 120 of the housing 110, allowing access to the pump subsystem 130 and computing device 150. In such aspects, the top portion 118 can be configured to be removable only by clinicians/doctors, and not by the subjects. Locking mechanisms requiring specialized tools can be utilized to prevent unauthorized access within the interior of the housing 110. Such configuration prevents subjects from manipulating the IPC device 20 to avoid fraudulent reporting for compliance monitoring, as well as preventing damage to the components within the housing 110. However, in other aspects, the top portion 118 can be removable without the need of tools. In such instances, the top portion 118 can be attached through a variety of means, including, but not limited to, tab-slot, friction fit, and the like.

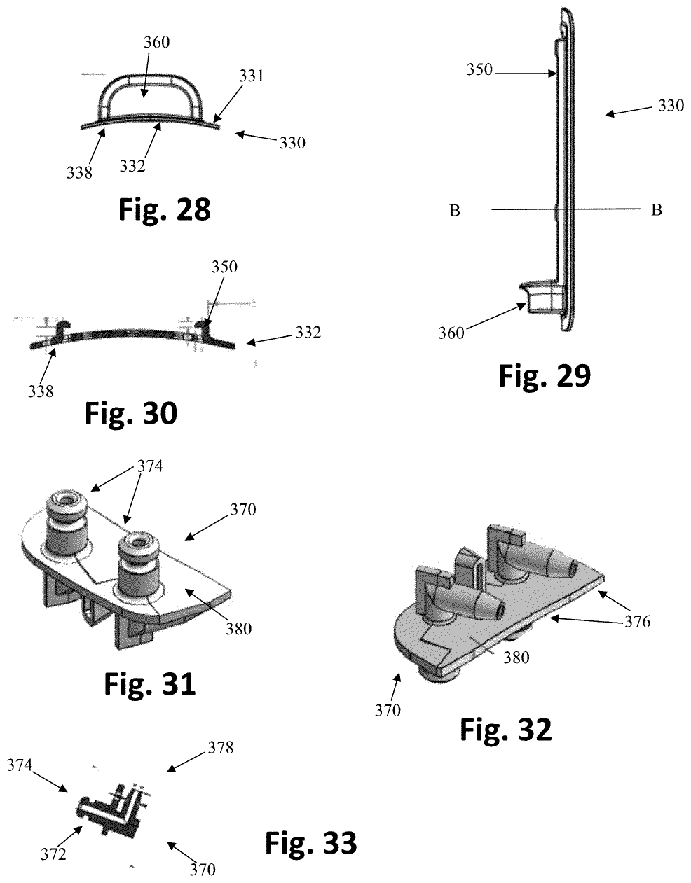

In another aspect, the body 111 of the housing 110 can ensure that the driving component 100 is mounted in the correct orientation and position on the inflatable sleeve 300, and more specifically, within a mounting means 330 attached to the inflatable sleeve 300, as shown in FIGS. 4, 7-10, 20-21, and 26-30. In such aspects, the mounting means 330 is configured to receive the housing 110 of the driving component 100. In an aspect, the mounting means 330 comprises a dock 330 The dock 330 provides a fluid interface for the driving component 100 and the internal components of the inflatable sleeve 300, discussed in detail below.

In an aspect, the dock 330 can be configured to be permanently attached to the inflatable sleeve 300. In such aspects, the dock 330 can be mounted via adhesives or other fastening means (e.g., RF) on a surface of the inflatable sleeve 300. In another aspect, the dock 330 can be configured to be removably attached to the sleeve 300. While various means of attachment can be used to secure the dock 330 to the inflatable sleeve 300, it is preferable that the dock 330 is attached in a manner that does not cause the subject discomfort. For example, if fasteners, permanent or removable, are used to attach the dock 330, it is preferable that such fasteners do not extend through the inflatable sleeve 300, but only extend through the top layer of the inflatable sleeve 300. In another aspect, the dock 330 includes a mounting extension/anchor portion 331, as shown in FIGS. 26-27. In such aspects, the mounting extension 331 can be configured to be received within the interior of the inflatable sleeve 300, covered by a fabric exterior. For example, as shown in FIG. 34, the inflatable sleeve 300 includes a fabric sleeve 302 with a top portion 304 and a bottom portion 306 that form a pocket 305 to receive the bladder 320. The dock 330 is attached to the bottom portion 306 and received within an opening 301 on the top portion 304 so as to be able to receive the driving component 100, and the mounting extension 331 being placed between the top portion 304 and bottom portion 306. In such aspects, the mounting extension 331 can be configured to be secured to interior components of the sleeve 300, including the sleeve itself and the inflatable bladder.

The dock 330 maintains a connection between the driving component 100 and the sleeve 300 that enables the driving component 100, and more specifically the pump subsystem 130, to communicate fluid pressure into a bladder 320 and then to the underlying tissues of the patient. When a bladder 320 is integrated into the sleeve 300, the dock 330 provides a fluid connection enabling the communication of fluid pressure from the driver into the bladder 320. When the bladder 320 is included as a integrated part of the driving component 100, the dock 330 provides the mechanical connection between the driver component 100 and the sleeve 300.

In an aspect, the dock 330 can have a shape that generally corresponds to the shape of the body 111 of the driving component 100, as shown in FIGS. 4-10, 20-21, and 26-30. In such aspects, the dock 330 includes a base member 332, a flange portion 350, and a dock extension 360. The base member 332 in general can match the overall shape of the body 111 of the driving component 100. The base member 332 can form the majority of the dock 330, and provides the fastening base for the dock 330 to be mounted to the inflatable sleeve 300 as discussed above. The base member 332 includes a top surface 334 and a bottom surface 336, with the top surface 334 configured to engage the driving component 100 and the bottom surface 336 configured to engage the inflatable sleeve 300. In an aspect, the bottom surface 336 can include a contoured surface 338. The contoured surface 338 is provided to increase the comfort of wear for the subject, the contoured surface 338 matching substantially the curvature of a subject's limb.

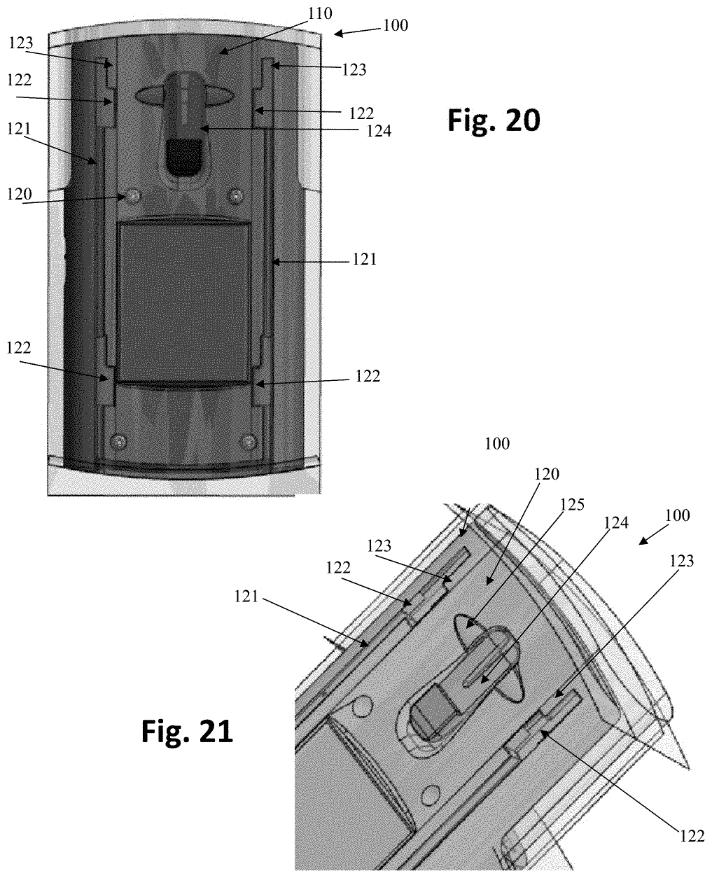

In an aspect, the flange portion 350 is configured to be received by a corresponding groove 121 found on the bottom portion 120 of the housing 110 of the driving component 100, as shown in FIGS. 20-21. In addition, securing tabs 352 can be found extending from the flange portion 350, that extend from the inner surface of the flange 350. The securing tabs 352 are configured to be received at notches 122 within the groove 121. Once the tabs 352 are received within the notches 122, the housing 110 can be advanced so that the tabs 352 are advanced in the groove 121, passing the notches 122, and retained under extensions 123 that reach into the groove 121. As shown, there are a total of 4 tabs 352, notches 122, and extensions 123. However, in other aspects, the numbers can vary. However, the number of tabs, notches, 122, and extensions should equal each other.

In addition, the base ember 332 of the dock 330 can include a receiving slot 340 configured to receive a latch 124 of the housing 110 of the driving component 100. In an aspect, the latch 124 can be configured to be spring loaded around a pivot mount 125, snapping into place in the receiving slot 340 when the driving component 100 is correctly aligned within the dock 330. By limiting the way in which the latch 124 can be received by the receiving slot 340, the orientation of the driving component 100 can be ensured when mounted onto the dock 330 of the inflatable sleeve 300. The latch 124 can be released by pressing the latch 124 from the back portion of the dock 330, through the fabric sleeve 302 in order to remove the driving component 100 from the dock 330. Such a configuration is not obvious to the user, and prevents removal of the driving component 100 while the sleeve 300 is on the subject.

The flange portion 350 can extend into a dock extension 360 located at a distal end of the dock 330. The dock extension 360 can be raised from the base 332, and can provide housing for a fluid interface 370 that contains fluid communication pathways 372 from the driving component 100 to the bladder 320 of the inflatable sleeve 300, as shown in FIGS. 31-33. In an aspect, the fluid communication pathways 372 include top connectors 374 to connect with the with the fluid pathway/communication ports 117 of the driving component 100 and bottom connectors 376 to connect to ports 326 of the bladder 320, via tubes 378 (see FIG. 34), putting the pump subsystem 130 in communication with the interior of the inflatable sleeve 300. The interface 370 includes a mounting base 380 configured to engage corresponding portions of the dock extension 360. In such aspects, the dock extension 360 includes ledge 363 for the sides of the mounting base 380 to engage and rest. In an aspect, the fluid communication pathway 372 forms a right angle between the top and bottom connectors 374, 376 connecting the driving component 100 to the inflatable balder 320.

In an aspect, the driving component 100 can be mounted to the inflatable sleeve 300, via the dock 330, in the following manner. First, the housing 110 of the driving component 100 is aligned with the general direction of the dock 330 of the inflatable sleeve 300. The tabs 352 of the flange 350 of the dock 330 are aligned and received within the notches 122 of the groove 121 of the housing 110. A distal end of the latch 124 of the housing 110 is inserted into the receiving slot 340 of the dock 330, and slid until the latch 124 snaps into position. As advanced, the extensions 123 of the groove 121 receives the tabs 352 of the flanges 350 of the dock 330. In addition, the fluid communication ports 117 are put in communication with the fluid communication pathways of the inflatable sleeve 300 via the interface 370 and its fluid communication pathway 372. To remove the driving component 100 from the inflatable sleeve 300, the latch 124 is compressed to disengage from the slot 340 of the dock 330 by pressing the latch 124 through the fabric sleeve 302, and the driving component 100 is pulled in the proximal direction so that the tabs 352 exits the notches 122 of the groove 121 of the housing 110.

The orientation of the housing 110 of the driving component 100 and mechanical locking aspects discussed above promote the proper orientation of the fluid communication ports 117 and pathway 372, such that the targeted treatment, compression emanating from the distal portion of the patient extremities, is facilitated. In other aspects, additional ports may be added to the IPC device 20 which would further necessitate the need for proper orienting and interaction of the components. These mechanisms (shape of body, extensions, and flexible tabs) provide an improvement to currently marketed devices which either do not provide a mechanism for attachment and detachment of the sleeve and IPCD devices, making re-use of the IPCD impossible, or do not provide orientation and indication in addition to securement. Especially for aspects of the IPC device 20 with multiple sensors, proper orientation, indication, and device securement may be required for reliable and intended function to occur.

In an aspect, the fluid interface 370 of the dock 330 can include a connector mechanism that seals the air pathways from fluids and particulates when the driving component 100 is not connected to the dock 330. In an aspect, the connector mechanism can includes valve bodies (e.g., one-way valves) that would prevent fluid contamination. The valve bodies would create a fluid-sealed state when the driving component 100 is not connected and establishes fluid communication with the driving component 100, and the pneumatic portions, with the sleeve 300/dock 330 when in a connected state. In such aspects, salve bodies would protect the interior of the sleeve 300, and the pathways 372, from particularly dirty environments when the driving component 100 is not in use. In addition, such valve bodies allow the sleeve 300 to be cleaned without fear of allowing fluid into the interior, allowing for longer uses by the user.

FIGS. 63-74 illustrate an IPC device 2020 according to another aspect of the present invention. As shown, the device 2020 includes a driving component 2100 and a sleeve 2300. As shown in FIG. 65, the sleeve 2300 can include anti-migration regions 2301 on an inner surface 2302 that prevents the sleeve from moving. Such regions 2301 can be made of materials that prevent slippage, including, but not limited to, silicon and the like. In such aspects, the driving component 2100 includes the same interior components as discussed above. The driving component 2100 includes a housing 2110 with a tapered body 2111 with a large end 2114, a small end 2116, a top portion 2118, and a bottom portion 2120. The small end 2116 can be configured to have fluid communication port(s) 2117 connected to the pump subsystem, and further configured to connect to the inflatable sleeve 2300. By having a tapered shape, the housing 2110 eliminates unnecessary space. Further, by tapering an end 2116 of the housing 2110, as well as smoothing out the edges along the top portion 2118 of the housing 2110, there is less chance of a sharp edge of the driving component 2100 getting caught on another surface, which could lead to damage as well as the driving component 2100 being forced off the sleeve 2300. In addition, the tapered body 2111 allows for the components of the driving component 2100 be oriented in such a way to save space and be as efficient as possible.

In an aspect, the bottom portion 2120 of the tapered body 2111 of the housing 2110 is configured to provide a base mount for the internal components of the driving component 2100. In another aspect, the tapered body 2111 of the housing 2110 can ensure that the driving component 2100 is mounted in the correct orientation and position on the inflatable sleeve 2300, and more specifically, within a mounting means 2330 attached to the inflatable sleeve 2300. In such aspects, the mounting means 2330 is configured to receive the tapered housing 2111 of the driving component 2110. In an aspect, the mounting means 2330 comprises a dock 2330. The dock 2330 provides a fluid interface for the driving component 2110 and the internal components of the inflatable sleeve 2300.

In an aspect, the dock 2330 can be configured to be attached to the inflatable sleeve 2300. In an aspect, the dock 2330 can be mounted via adhesives or other fastening means on a top surface of the inflatable sleeve 2300. In an aspect, the dock 2330 includes a mounting extension 2331, as shown in FIGS. 74 & 74B. In such aspects, the mounting extension 2331 can be configured to be received within the interior of the inflatable sleeve 2300, covered by a fabric exterior discussed in detail below. In such aspects, the mounting extension 2331 can be configured to be secured to interior components of the inflatable sleeve and the fabric exterior.

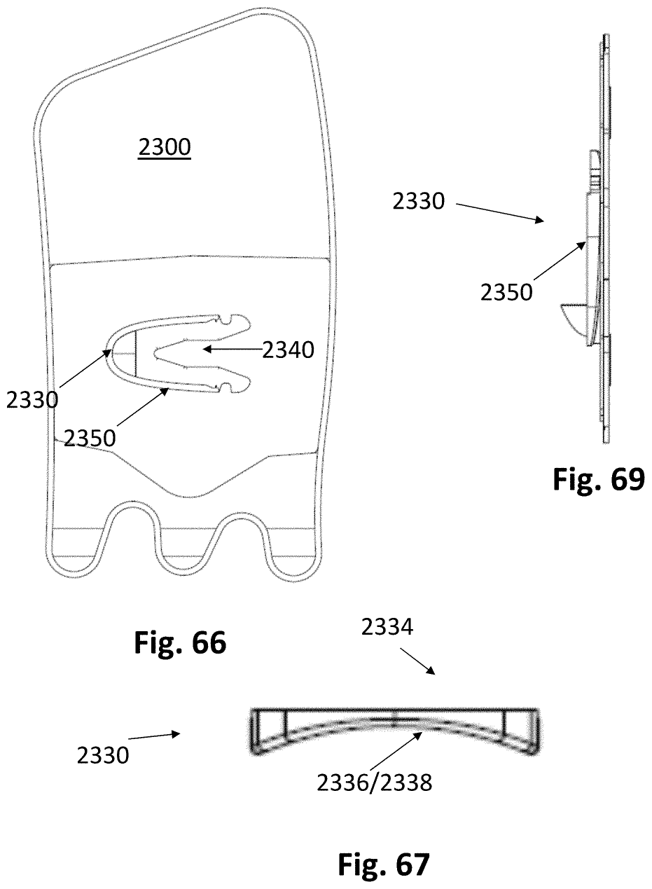

In an aspect, the dock 2330 can have a shape that generally corresponds to the shape of the tapered body 2111 of the driving component 2110, as shown in FIGS. 66-69. In such aspects, the dock 2330 includes a base member 2332, a flange portion 2350, and a dock extension 2360. The base member 2332 in general can match the overall shape of the tapered body 2111 of the driving component 2100. The base member 2332 can form the majority of the dock 2330, and provides the fastening base for the dock 2330 to be mounted to the inflatable sleeve 2300 as discussed above. The base member 2332 includes a top surface 2334 and a bottom surface 2336, with the top surface 2334 configured to engage the driving component 2110 and the bottom surface 2336 configured to engage the inflatable sleeve 2300. In an aspect, the bottom surface 2336 can include a contoured surface 2338. The contoured surface 2338 is provided to increase the comfort of wear for the subject, the contoured surface 2338 matching substantially the curvature of a subject's limb.

The base member 2332 can also include a receiving slot 2340 that is configured to receive a matching tab/extension 2122 of the housing 2111 of the driving component 2110. The receiving slot/groove 2340 includes a distal end 2341 and a proximal end 2342, the proximal end 2342 oriented such that it is closer to the torso of the subject than the distal end 2341 when the inflatable sleeve 2300 is mounted on a limb of the subject. In an aspect, the proximal end 2342 includes an opening 2343 for the groove 2340, with the groove 2340 including a closed end 2344. In an aspect, the receiving slot 2340 is shaped so that there is only one way in which the tab/extension 2122 of the housing 2111 can be received. For example, as shown in FIGS. 10-12 and 22-23, the groove 2340 can have a dovetail shape at the closed end 2344, which matches a dovetail shape of the tab/extension 2122 of the housing 2110 of the driving component 2100. By limiting the way in which the tab 2122 can be received by the groove 2340, the orientation of the driving component 2110 can be ensured when mounted onto the dock 2330 of the inflatable sleeve 2300. In addition, edges of the groove 2340 can be configured to form flanges 2345 that can be received within a corresponding slot 2123 found on the tab 2122 of the housing 2110 of the driving component 2100. The flange/slot combination further secures the driving component 2100 into the dock 2330 of the inflatable sleeve 2300, ensuring the driving component 2100 is oriented in the correct direction.

In an aspect, as discussed above and shown in FIGS. 66 and 68-69, the dock 2330 includes a flange portion 2350 that can extend upwards from the base portion 2332 of the dock 2330. The flange portion 2350 can engage the edges of the tapered body 2111 of the housing of the driving component 2100. The flange portion 2350 can include a proximal portion 2352 adjacent to notches 2353, in an aspect, the housing 2110 of the driving component 2110 includes flexible tabs 2124 that are configured to be received by the notches. In an exemplary aspect, the flexible tabs 2124 include a compression portion 2125 and a connecting portion 2126. The connecting portion 2126 are connected to the base 2120 of the housing 2110 of the driving component 2100, with the compression portions 2125 being able to move in a limited fashion opposite the connecting portion 2126. The bottom portion 2120 of the housing component 2110 can include recesses 2127 that house the flexible tabs 2124. When compression is applied to the compression portions 2125, the compression portions 2125 are received fully within the recesses 2127. When no compression is applied to the compression portions 2125, the compression portions 2125 extend beyond the recesses 2127, and out beyond the profile of the housing 2110. Notches 2128 can be found along the compression portions 2125, which correspond to tabs (not shown) found on the flange portion 2350.

The flange portion 2350 can extend into a dock extension 2360 located at a distal end of the dock 2330. The dock extension 2360 can be raised from the base 2332, and can provide housing, as well as a fluid interface, for fluid communication pathways of the inflatable sleeve 2300. In an aspect, the dock extension 2360 includes fluid pathway connectors 2362 which match with the fluid pathway ports 2117 of the driving component 2100, putting the pump subsystem 2130 in communication with the interior of the inflatable sleeve 2300.

In an aspect, the driving component 2100 can be mounted to the inflatable sleeve 2300 in the following manner. First, the housing 2110 of the driving component 2100 is aligned with the general direction of the dock 2330 of the inflatable sleeve 2300. A distal end of the tab/extension 2122 of the housing 2110 is inserted at the proximal end 2342 of the receiving groove/slot 2340 of the dock 2330, and slid until the proximal end 2342 reaches the distal end 2344 of the groove 2340. As advanced, the slot 2123 of the tab/extension 2122 receives the flanges 2345 of the dock 2330. As the housing 2110 is advanced, the flange portion 2350 of the dock 2330 apply forces/compresses the compression portions 2125 of the flexible tabs 2124, pushing them into the recesses 2127, until the resting position of the housing 2110 within the dock 2330 is reached. At this point, the flexible tabs 2124 are no longer compressed by the flange 2350, and can extend so that the notches 2128 of the tabs 2124 can engage tabs of the flange portion 2350. In addition, the fluid communication ports 2117 are put in communication with the fluid communication pathways of the inflatable sleeve 2300 (see FIG. 74B). To remove the driving component 2100 from the inflatable sleeve 2300, the flexible tabs 2124 are compressed to disengage from the flange 2350 of the dock 2330, and the driving component 2100 is pulled in the proximal direction so that the tab 2122 exits the opening 2343 of the receiving slot 2340.

The taper and mechanical locking aspects discussed above promote the proper orientation of the fluid communication ports 2117, 2362, such that the targeted treatment, compression emanating from the distal portion of the patient extremities, is facilitated. In other aspects, additional ports may be added to the IPC device 2020 which would further necessitate the need for proper orienting and interaction of the components. These mechanisms (shape of body, extensions, and flexible tabs) provide an improvement to currently marketed devices which either do not provide a mechanism for attachment and detachment of the sleeve and IPCD devices, making re-use of the IPCD impossible, or do not provide orientation and indication in addition to securement. Especially for aspects of the IPC device 20 with multiple sensors, proper orientation, indication, and device securement may be required for reliable and intended function to occur.

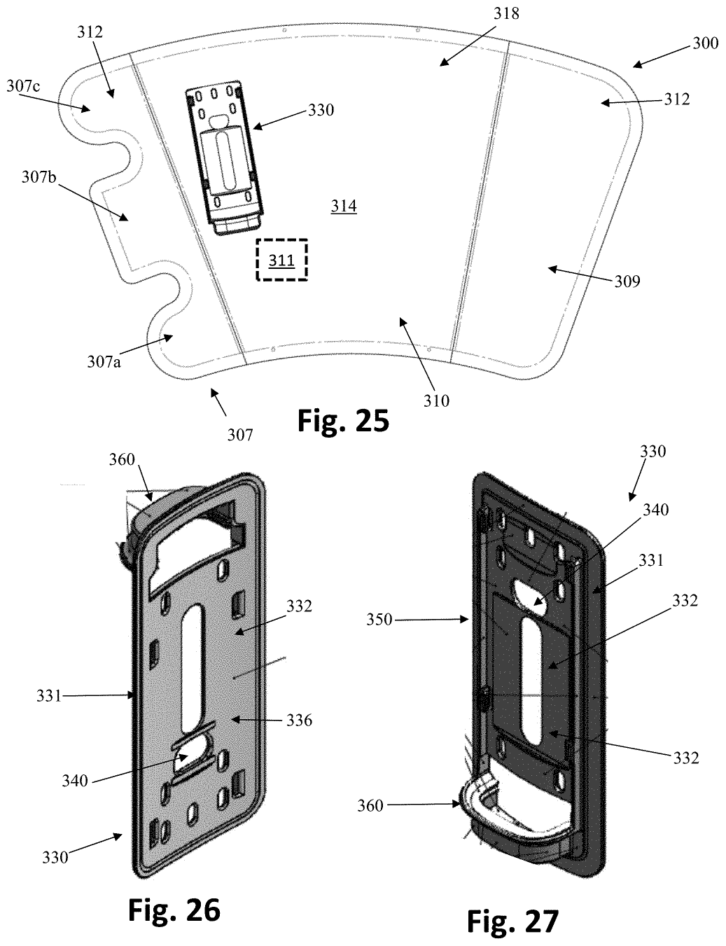

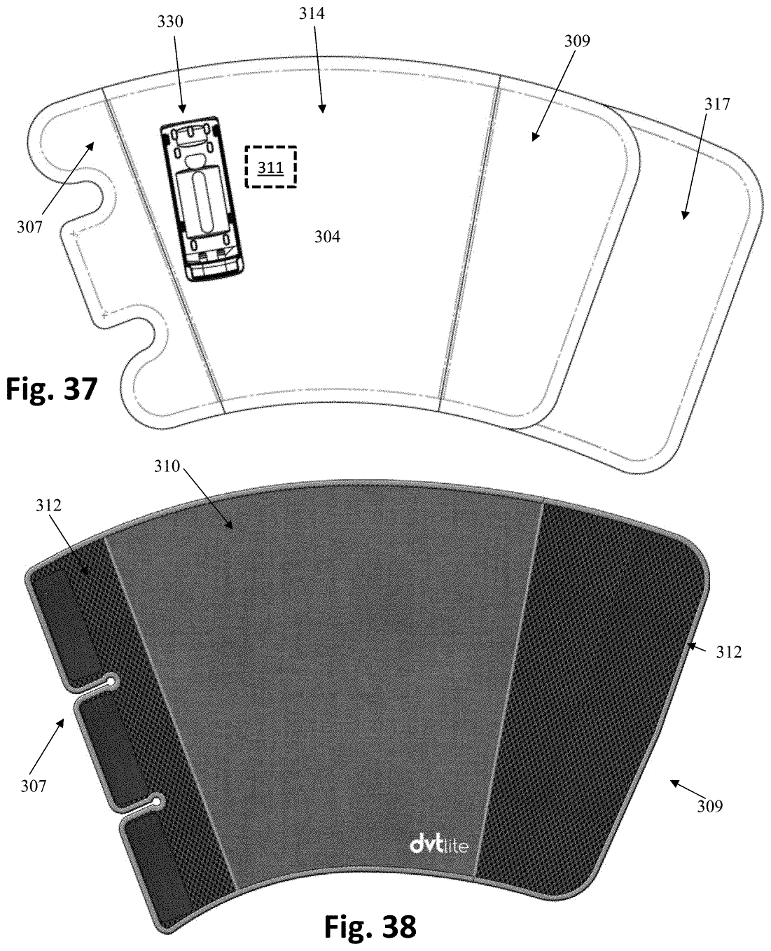

In an aspect, the sleeve 300 can include a unique identifying means 311, as represented in FIGS. 25 and 37. In an exemplary aspect, the unique identifying means includes a RFID chip 311 with a unique identifier specific to that sleeve 300. The RFID chip 311 can be mounted onto or inserted into the fabric sleeve 302 of the sleeve 300, or into the dock 330 on the sleeve 300. In such instances, the driving component 100 can include an RFID scanner (not shown) with the ability to communicate with other computing devices. In another aspect, the sleeve 300 can include an indicator to illustrate the location of the RFID chip 311 so that clinicians can scan the RFID chip 311 when assigning a sleeve 300 to a patient, a driving component 100 to the patient, or a power source 180.

The combination of the unique identifying means associated with the sleeve 300, and not the driving component 100, and the RFID scanner, possibly with the driving component 100, allows information to be reported for the patient—that is the only user of the sleeve 300 as assigned. In such instances, when the patient is first prescribed DVT treatment, a sleeve 300 is registered to that patient. By assigning the sleeve 300 to the patient, any driving component 100 of the IPC device 20 can be used with the sleeve 300, with the driving component 100 identifying the sleeve 300 and associating the unique identifier with the data that the driving component 100 collects during use. The data of the patient, associated with the unique sleeve identifier, can then be transferred to a centralized data center, which can store the data in a database. In such instances, patient compliance can be tracked regardless of which driving component 100 is used because the unique sleeve identifier data would be reported to a central database by any number of driving components 100. This allows hospitals and other large user groups to track individual patient use and compliance data without requiring specific patients to be assigned to the removable driving components 100. Such an aspect is particularly valuable if the driving component 100 does not include a removable battery and recharging a patient-specific component would prevent collection of patient use data.

In addition, by having the patient data linked to a sleeve 300 and not the driving component 100, and having the driving component 100 communicate such data to a centralized repository, the driving component 100, and its computing device 150, including processing means and memory, can be configured to only temporarily retain the patient data. That is, the computing means 150 of the driving component 100 can be configured such that data obtained from the patient, identified by the sleeve identifier, is only stored during the use of the driving component 100 of the patient—after communicating the data throughout use, or at its completion, the patient data is deleted from the computing means 150 of the driving component 100, either automatically (e.g., detachment from sleeve, removal of battery, or placing into charging station), or manually by the caretaker/physician/nurse (via the computer or through interface on component 100). This allows for smaller memory needs and protects the privacy of the patient's data.

In addition, by utilizing a unique device identifier for the sleeve 300 in combination with the driving component 100, it is possible to track additional information. For example, the system 10 can be set up to prevent a sleeve 300 from being recycled. For example, once the sleeve 300 has been assigned to a specific person, the system 10 can be configured to prevent that sleeve 300 from being used with another individual. For example, when a driving component 100 is assigned to patient, the driving component 100 can confirm that the sleeve 300 corresponds to the assigned patient on record. If not, the driving component 100 can alert the system 10, which can then alert an administrator to the problem. In addition, the unique sleeve identifier can be used to prevent a patient from using the wrong driving component based upon their needs, or keep the patient assigned to a specific type or individual driving component 100.

As discussed above, the housing 110 of the driving component 100 contains a pump subsystem 130, as shown in FIGS. 16-19. The pump subsystem 130 includes a fluid pump 132 and a valve/solenoid 134, connected by a flow tube 136. The valve/solenoid 134 controls the fluid (e.g., pressurized air) from the fluid pump 132 through additional tubing 138 to fluid pathway ports 117 of the housing 110, which ultimately connect to have fluid communication with the bladder 320, discussed in more detail below.

In an aspect, the fluid pump 132 can comprise a pneumatic pump 132. The pneumatic pump 132 can be a standard use pneumatic pump 132 found in other DVT treatment devices. The pneumatic pump 132 must be powerful enough to provide the needed pressure for applying the compression needed to prevent DVT. In such aspects, the pump 132 should be able to operate at therapeutically appropriate ranges, and should be able to deliver no less than 55 mmHg pressure. The pump 132 and surrounding casing should also minimize and noise/vibration caused by the use of the pump 132. Pump weight and physical size are also minimized to reduce the overall devices weight and bulk. Given this is a wearable device, these considerations are particularly important as they reduce the patient burden to wear the technology and therefore comply with clinically recommended treatment.

The pneumatic pump 132 is in communication with fluid pathway flow ports 117 found within the housing 110. The fluid pathway ports 117 are configured to be in communication with the corresponding air flow pathway(s) 372 found in the mounting means 330 of the inflatable sleeve 300, discussed in detail below. In an aspect, the pneumatic pump 132 is configured to pump air into the inflatable sleeve 300 through one fluid pathway port 117 to the corresponding fluid communication pathway 372 of dock 330 and then to the inflatable sleeve 300. Other matching air flow ports can be configured to monitor the air flow through an air flow sensor.

In an aspect, as shown in FIGS. 16-19, the pneumatic pump 132 is controlled by the computing device 150. In an aspect, the computing device 150 includes a processor, memory, data storage means, and additional known computing components. In addition, the computing device 150 is configured to communicate with various sensors found within the driving component 100. Such sensors include, but are not limited to, air flow, temperature, movement, and the like. Sensors used could include, temperature sensors (to detect the presence of a patient), probes, gyroscopic or other motion trackers (to aid in the detection of patient activity), radio-frequency identification tags (RFID) to allow for pairing of the pump-housing device to a particular sleeve 300 (anatomical region, etc.), or global positioning system (GPS) sensors to enable real time tracking of the device 20. The computing device 150 can include a processor, memory, sensors, and communication means, as well as other components known to be a part of smaller computing devices 150. In an aspect, the processor is configured to work with programs that control the operation of the pneumatic pump 132 of the subsystem 130. The computing device 150 activates and deactivates the pneumatic pump 132 to apply the compression at needed intervals. Further, the computing device 150 can control the valve/solenoid 134 to reduce inflatable regions within the inflatable sleeve 300. The pressure reduction is done through the selective activation of the valve/solenoid 134, which vents the contained pressure held within the inflatable region of the sleeve with the outside atmosphere. A power switch 160 powers on and off the components (computing device 150 and pump subsystem 130) of the driving component 100.

In an aspect, the pump subsystem 130, controlled by the computing device 150, is configured to be able to fill the inflatable sleeve 300 starting in a peristaltic fashion, and be able to achieve a maximum pressure of 55 mHg at the sleeve 300 at the top end. In addition, in some aspects, the maximum pressure is achieved in 10 to 20 seconds, while being able to maintain the highest pressure for up to 5 seconds. In an aspect, the computing device 150 can be configured to apply the compression cyclically for predetermined times at predetermined intervals. In an aspect, the system can be configured to operate on inflation and deflation cycles that occur in less than 60 second intervals. In addition, the computing device 150 is in communication with the sensors, from which the computing devices 150 collects information about the activity of the driving component 100. The sensors can measure the time, amount of pressure, the frequency, and temperature of the subject in order to ensure that the subject is complying with the prescribed treatment program.

In an aspect, the computing device 150 can act as a meter for the patient sticking to the prescribed treatment plan. For example, the computing device 150 can be configured to measure the daily compliance of a user. For example, if a patient is prescribed to use the IPC device 20 for 18 hours a day, the computing device 150 can communicate with the pneumatic pump 132 and sensors to track the time of active use of the device 20. In an aspect, the computing device 150 can also track patient compliance, or the fractional number of days over the treatment term in which the subject has been in 100% daily compliance. In an aspect, the computing device 150 can be configured to store patient data for the duration of the prescribed treatment. In such aspects, the patient data, including compliance, can be stored from 1 day up to 90 days, if desired. As discussed above, the patient data can be uploaded from the computing device 150 to a server 70 associated with the system 10 either through a wired connection or a wireless connection over a network 50 via various radio transmitters known in the art.

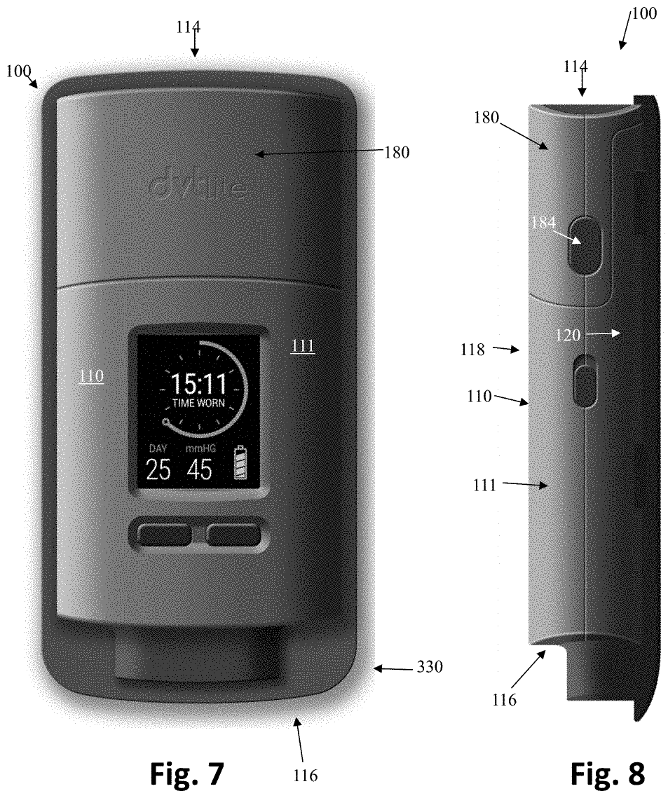

In another aspect, the computing device 150 can include a user interface 152. The user interface 152 can include a graphical user interface display 153 (e.g., made of a combination of a LCD screen 153a and a window 153b) and input devices 154 (e.g., buttons in communication with the remainder of the computing device 150, with corresponding inlets in the housing 110 of the top portion 118) that communicates information to the subject, as well as taking input from the user (cycling through information, clearing messages, etc.), as shown in FIGS. 7, 11-12, and 19. For example, the user interface 152 can report to the user whether or not the IPC device 20 is active, the power level of the device 20 (e.g., the time left or amount of power left on the self-contained power source 180), whether it is applying pressure or deflating, the amount of pressure being applied, how much time is left in a pressure cycle, the number of cycles completed in a day, how much treatment time is still needed for a day, the power s well as the daily compliance and patient compliance numbers discussed above. In addition, the GUI 152 can report to the user or administrator other various messages known to the system. For example, if the prescribed treatment length assigned to the user has been obtained, a notice of completion can be displayed. For example, if the prescribed time was 34 days of treatment, a notice can be displayed on the GUI 152 to indicate completion at 34 days. In other aspects, if the prescribed length has been extended, or use by the patient has been extended, messages can be provided to the user and the administrator to see if extended time is needed. For example, if the prescribed time is 34 days, and a patient is subjected to a 35^{th} day, the system check to see if an extended prescription period is needed.

In an aspect, the user interface 152 can be configured to only display information to the subject, and not let the subject control the operation of the IPC device 20. In an aspect, the IPC device 20 is configured such that only clinical service providers that are trained on the system would be able to reset the operations of the computing device 150. Messages for the providers can also be displayed (e.g., reset the device for a new patient). In such aspects, the provider can reset the compliance meters of the computing device 150. In another aspect, the IPC device 20 can include a remote application that can operate on an individual's local computer or smart phone, delivering the information that would be delivered on a local user interface, with the IPC device communicating via wireless means, discussed below.

In an aspect, the computing device 150 can be configured to have communication means configured to communicate with other devices. For example, the computing device can include radio chips (e.g., Wi-Fi, Bluetooth, etc.) that communicate with other computing devices. In such aspects, the IPC system 10 can include a server (not shown) that is configured to communicate with the individual IPC devices 20 in order to capture patient data, including compliance data. In one aspect, the server is configured to communicate with electronic healthcare records, such that the data collected on the IPC device 20, where appropriate, can be easily entered copied onto a patient's healthcare record. In an aspect, the system 10 can also be configured to provide incentives to subjects for staying in compliance with the prescribed treatment. For example, the system 10 can be gamified, which would remind the subject about staying in compliance as well as rewarding such subjects for compliance. This can be reinforced through sharing messages through the display graphic/user interface. In one configuration, the IPC device 20 may also be paired with an application which would be able to display information about compliance, compliance trends, clinical notification, and the like to either the patient using the system or the care provider responsible for monitoring the patient.

In an aspect, the mobile IPC device 20 is powered by a self-contained power source 180. In an aspect, the power source 180 can include a removable rechargeable battery 180. In an exemplary aspect, the rechargeable battery 180 includes a lithium ion battery. The power source 180 powers the computing device 150, the sensors, and the pump subsystem 130. In an aspect, the power source 180 is configured to run the IPC device 20 for 18 to 24 hours. In an exemplary aspect, the battery 180 can be a 22400 mAh battery, which allows for battery life over twenty hours. However, in aspects where the IPC device 20 is prescribed for continuous use (e.g., 24 hours a day), the power source 180 can include multiple rechargeable batteries 180 that are used to replace each other when their power runs out to ensure continuous operation of the IPC device 20.

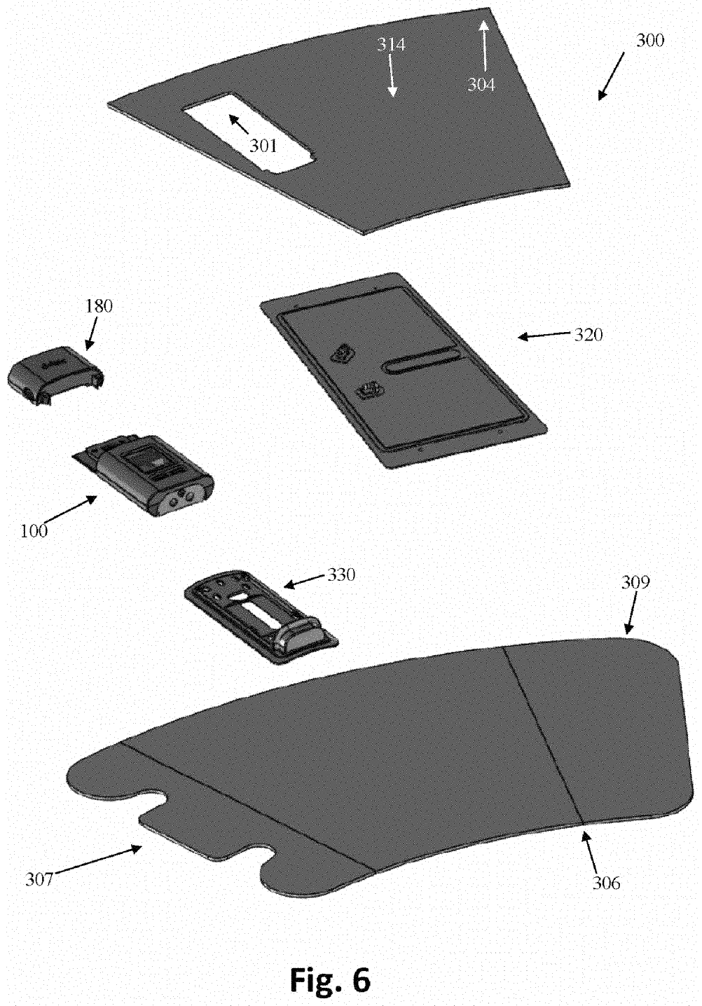

In an aspect, the power source 180 is configured to be removed from the driving component 100, as shown in FIGS. 6-18 and 22-24. In an exemplary aspect, the power source 180 includes separate housing 182 that is configured to removably, yet securely, engage the housing 110 of the driving component 100. The combination of the housing 110 and the power source 180 are configured to enable removable attachment of the power source 180 so it can be recharged separately in a recharging system, as shown in FIGS. 41-44. This embodiment enables creation of an IPC system 10 that is never attached to a grounded power supply during use, thereby avoiding the risks of wall power and electrically grounding the user. In addition, switches 184 can fully disconnect the battery 180 from the driving component 100.

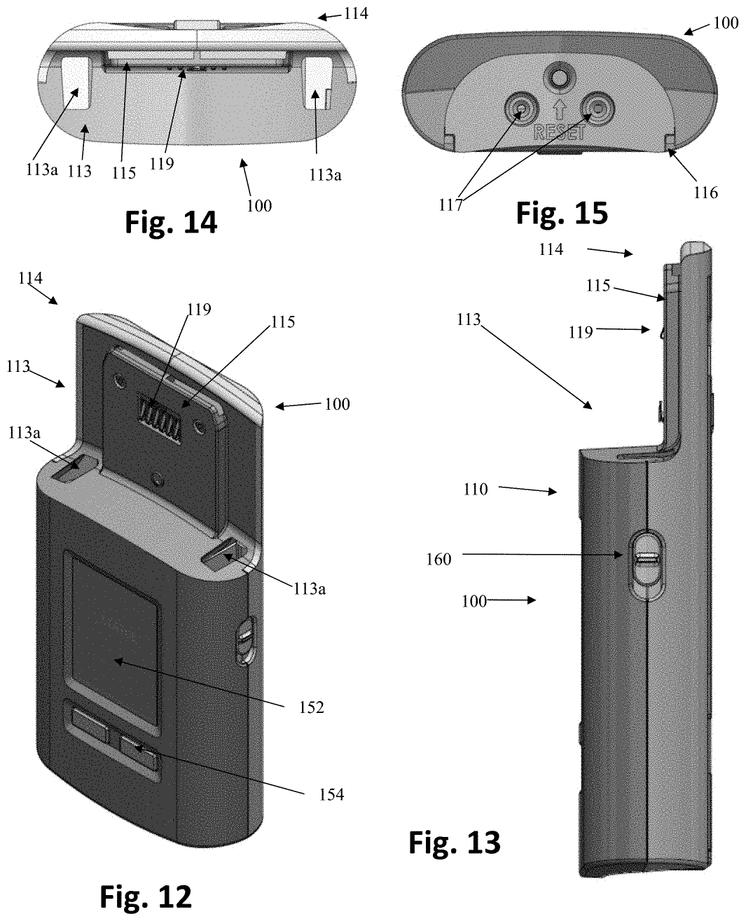

In an aspect, the power source/battery housing 182 is configured to have a battery connector 186 that engages a corresponding battery connector 162 of the driving component 100. As shown in FIGS. 12-14, the first end 114 of the housing 110 can include a recessed portion 113 that matches the shape of the housing 182 of the battery 180. In addition, an extension 115 can be found that holds the battery connector 119 of the driving component 100. The battery housing 182 can include an inlet 183 that matches the extension 115 is shape, and also includes the battery connector 186, to ensure a good connection between the battery connectors 119, 186. In addition, the battery housing 182 can include securing means 188 that are used to secure the housing 182 to the housing 110 of the driving component 100. For example, the securing means 188 can include, but are not limited to, releasable tabs 188 that engage receiving slots 113a in the housing 110 of the driving component. The switches 184 can release the tabs 188 from the receiving slots for removal of the battery 180. In an aspect, the switch 184 can include two switches 184, or compression releases 184, that activate the releasable tabs 188 for removal from the receiving slots 113a of the housing 110 of the driving component 100.

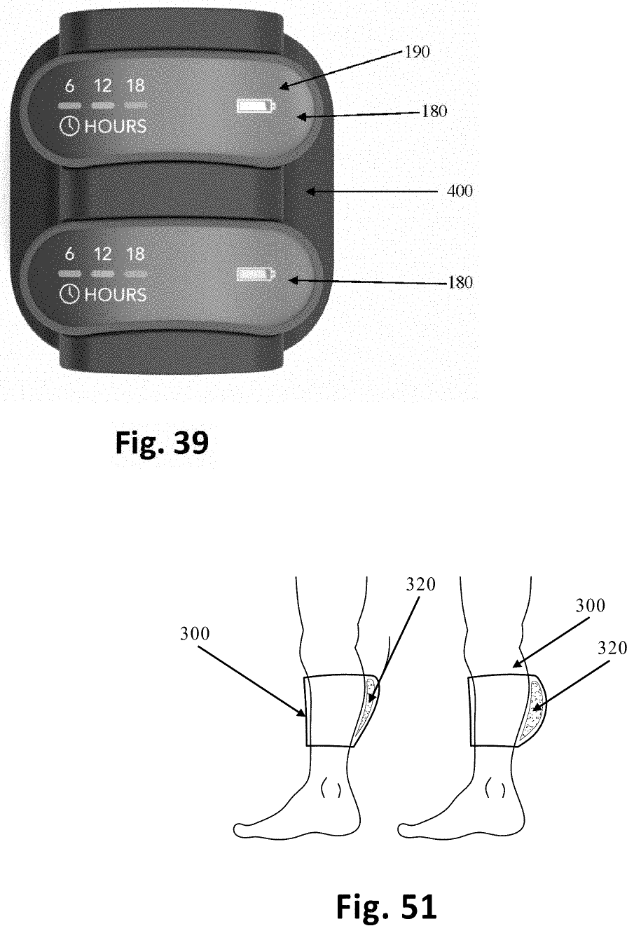

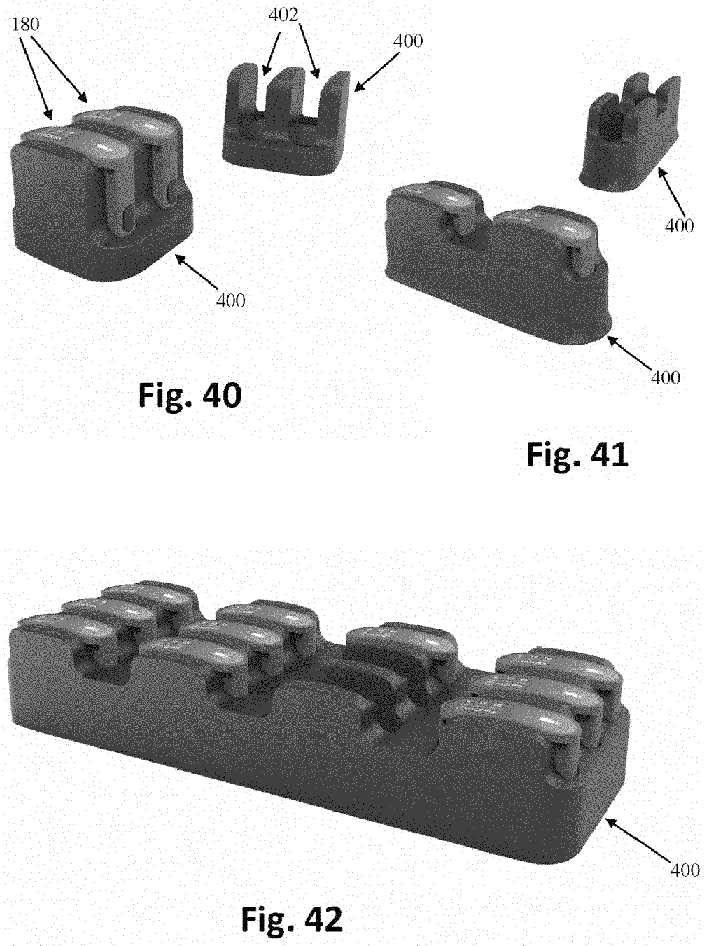

The battery 180 can also include computing means (not shown) that monitors the power level of the battery. In an aspect, the battery includes a display 190 to inform the used of the power level of the battery 180. In an aspect, the IPC system includes a battery charger 400, as shown in FIGS. 39-42. The battery charger 400 can include multiple charging ports 402 to receive multiple batteries 180.

In an aspect, the battery 180 can only be charged when removed from the IPC device 20. In such aspects, the mobile IPC device 20 can be configured so that it cannot be powered without any other power source than a rechargeable battery 180. In other words, the IPC device 20 does not function in the absence of the battery 180, and the battery 180 can only be charged when it is removed from the IPC device 20. In an aspect, the battery 180 is mechanically design to contain recharging features that are only accessible when not attached to the driving component 100. In one embodiment, the physical plug connection 186 is only accessible when the battery 180 is removed from the driving component 100. In another aspect, the battery is charged using inductance, and the inductance antennae 186 is only accessible for charging when the battery is removed from the driving component 100, so that the battery 180 can only be charged in a solitary state.

While in the preferred embodiments of the invention, a battery 180 will provide the power needed for the entire prescribed treatment plan (e.g., 18 hours). Upon completion of the treatment plan, the battery 180 is removed and recharged, discussed below. Another charged battery 180 can be inserted to continue operations of the IPC device 20. In an aspect, the mobile IPC device 20 can be configured to monitor the power level of the battery via the display or through communication means.

In an aspect, the mobile IPC device 20 of the IPC system 10 is configured to provide sufficient pressure to extremities, as well as removal of such pressure, in a periodic manner. In such aspects, the mobile IPC device 20 loads and unloads a pre-defined pressure value through a loop of pre-defined duration. In an aspect, the pressure applied is at least 55 mmHg, which was determined through a clinical literature review and evaluation as the required pressure to decrease venous stasis. The loop must allow for venous refill in between compression cycles, which has been found in clinical literature to be approximately 20-30 seconds. In one aspect, programming of the device 20 is controlled through firmware, and cannot be modified or tailored. In another embodiment, RFID tags may be utilized as a means to couple a sleeve 300 and the driving component 100, and may also be used as a method to select parameters about the treatment. In this aspect the RFID code may change settings corresponding to the pressure profile, compliance durations, etc. These MD systems may also correspond to a particular anatomic region, to which the above mentioned device settings could be tailored.

As discussed above, the inflatable sleeve 300 of the IPC device 20 is configured to engage the limb of a subject. In an aspect, the inflatable sleeve 300 is configured to fit a patient's limb comfortably and enough to fully secure the sleeve 300 on the limb. In such aspects, the sleeve 300 is configured to fit a wide variety of limbs of subjects. In an aspect, the sleeve 300 is comprised of a closed circle of material that slides over the patient's anatomy and closes to accommodate the anatomy. In an aspect, the sleeve 300 has a cylindrical shape, with openings at either end, to fit an individual's appendage. In an aspect, the sleeve 300 can have an increased diameter from one end to the other to accommodate appendages of individuals.

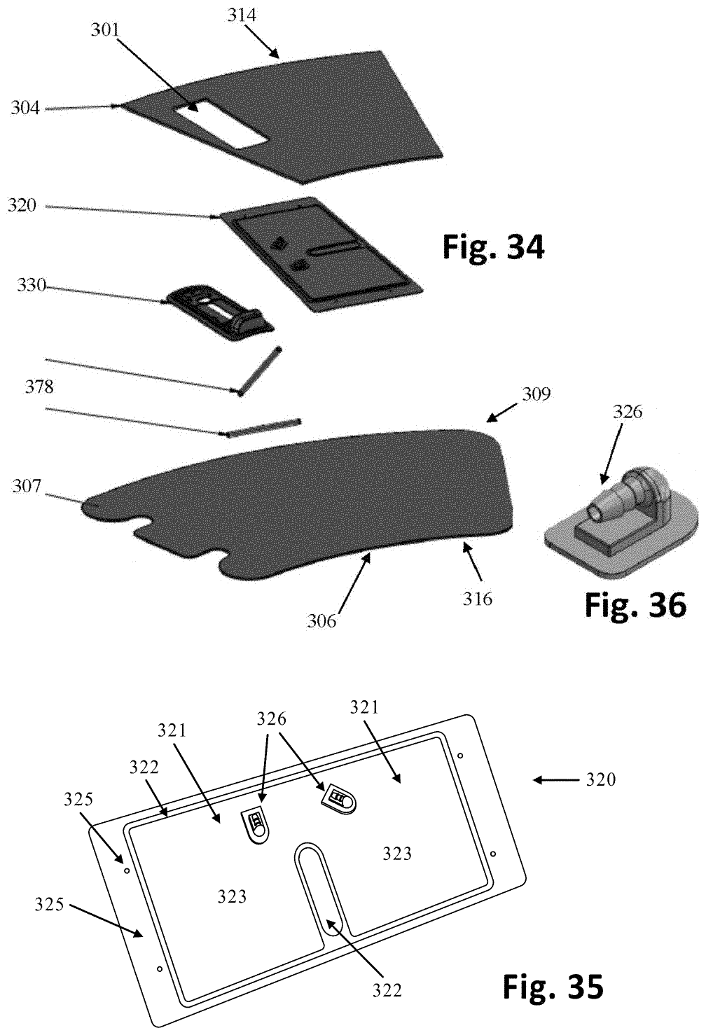

In an aspect, the inflatable sleeve 300 incudes a textile/fabric sleeve portion 302 and an air-impermeable bag portion/bladder 320, the textile portion 302 encompassing the impermeable bag portion/bladder 320, as shown in FIGS. 4-6 and 34-38. In an aspect, the textile portion 302 includes a top portion 304 and a bottom portion 306 that are connected to one another and form a pocket/containing portion 305 for the bladder 320. In an aspect, the bladder 320 is made of an air-impenetrable material that forms channels 321 for air to inflate and deflate. The bladder 320 can include a seal 322 that defines the ends of the inflatable portion 323. A salvage edge 324 of the material can extend from the seal 322. The salvage edge 324 can be utilized for mounting purposes, discussed in more detail below. In an aspect, the salvage edge 324 can include apertures 325 to receive mounting pins when placed within the pocket/retaining portion 305 of the sleeve 300. In addition, the sleeve 300 includes air flow ports 326. The air flow ports 326 connect to the interface 370 via tubes 378. In an aspect, the bladder 320 can include a central weld 328 that assists in forming channels 321.

In such aspects, the textile portion 302 is configured to enable the conduction of heat and moisture from the sleeve-skin interface during us while avoiding any chemical or physical irritation of the user's skin. In an aspect, the textile portion 302 can include porous materials that allow breathability; that is, the material enables ambient air to remove heat and moisture from the skin and sleeve 300 using conduction, convection, and radiation. Such porous materials include, but are not limited to, permeable felts and fabrics, open-cell foams, and composites or laminates of the two materials. Another embodiment would be an otherwise impermeable material with perforations made within the material to create porosity. In other aspects, non-porous materials like polyvinyl chloride or polyester sheets can be used, but with macro-pores added to the material. In an aspect, the textile portion 302 comprises a chemical composition that is in a biocompatibility with the skin of the user. In an aspect, the textile portion 302 is configured to be absent of mechanical features or sources of potential irritation on areas of the sleeve that are in direct contact with the user. (protrusions, abrasions, sharp edges, hard materials, hard edges, geometric features, etc.). In some instances, removal of mechanical irritations requires the use of buffer materials, piping, stand-offs, ribbons, or the like.

In an aspect, the textile portion 302 is a composite material system, made of a combination of a stiff fabric 310 that has little stretch and an elastic/flexible fabric 312. By utilizing a combination of materials, the sleeve 300 is able to conform to the complex user anatomy (compliant enough) but maintain sufficient mechanical integrity (rigid enough) to hold the bladder in place during use and convey mechanical energy into the underling tissues, as shown in FIG. 51. The stiff fabric 310 in configured to have little stretch when a mechanical load is applied. Such stiff fabrics 310 can include, but not are limited to, cottons, PET woven or non-woven sheets, nylon meshes, and the like. The elastic fabric 312 is configured for optimization of comfort and breathability. Such elastic fabrics 312 can include, but are not limited to, elastic fabrics like Lycra, cottons with elastic filaments, foams, loose felts, or woven filaments. The stiff fabric 310 is configured for use around the pocket/containing portion 305 to contain the bladder 320 to ensure the bladder energy during inflation is directed towards the underlying tissues intended to be treated. The stiff fabric 310 is also used as a mechanical mounting location for the driving component 100 and the mount 330 discussed above. The driving component 100 requires a mechanically robust location on the sleeve 300 because of the weight and the expected mechanical forces applied by driving component 100 on the underlying stiff fabric 310 during user ambulation.

In an aspect, the textile portion 302 is made from a soft textile for the comfort of the patient, such as silicone foam or the like. In one configuration this material can be easily cleanable, or anti-microbial. In an aspect, the textile portion 302 can be configured to include a containing portion 305 that contains the air-impermeable bag/bladder 320, and securing portions 307, 309 that are used to secure the inflatable sleeve 300 on the limb of the subject. In such aspects, the securing portions 307, 309 are found on opposite portions of the containing portion 305. The containing portion 305 fully separates the air-impermeable bag 320 from the securing portions 307, 309, which contain enough fabric to allow for adjustable use of the sleeve to accommodate a variety of sizes of limbs. In an aspect, a sleeve extension 317 can be utilized to connect to the securing portions 307, 309 for patients with larger appendages. In an exemplary aspect, the securing portions 307, 309 make up connectable ends of the inflatable 300. In an aspect, various connecting means, including, but not limited to, hook and loop fasteners, button fasteners, tab and slot fasteners, and the like, can be used to secure the securing portions 307, 309 of the sleeve 300 to one another, which allows the sleeve 300 to be adjustable to the size of the limb of the subject

In an aspect, the sleeve 300 is configured so that the patient/user can easily mount the sleeve 300 on the patient's appendage. In an aspect, the sleeve includes an attachment mechanism. In an aspect, the attachment mechanism can utilize securing portions 307, 309 of the fabric sleeve 302. In an exemplary aspect, the securing portions 307, 309 employ a hook and loop attachment system, the securing portions 307, 309 including a hook portion and a loop portion. The hook portion and the loop portions are configured to be placed on opposite surfaces and opposite ends of the sleeve securing portions 307, 309 of the fabric sleeve 302 (e.g., top left and bottom right of the sleeve or vice versa) as the sleeve 300 forms a tube when secured on the limb of the subject. The hook portion includes a plurality of hooks, and the loop portion includes a plurality of loops, with the loop portion configured to receive the hook portion. In an aspect, the securing portions 307, 309 can be comprised of a fabric that acts like loop portions, therein only needed hook portions added to a surface. The hook and loop portions can be attached to the sleeve 300 via adhesion, welding, sewing, melted, chemically or otherwise, and other known methods of connection. In other embodiments, other securing mechanisms, including, but not limited to, laces, zippers, buttons, snaps, compression fits, etc., can be utilized. Hooks and loop fasteners, however, offer a continuously variable connection and are convenient.

In one aspect, the securing portions 307, 309 can include one large hook portion and one large loop portion. In another aspect, multiple corresponding hook and loop portions, of matching sizes, can be used, as shown in FIGS. 34 and 37-38. These different portions can be thought of as toes. In an aspect, two toes can be utilized. In another, three toes 307a-c can be utilized. In such aspects, the toes can be labeled to indicate to the user which order the toes should be secured during placement on the patient. In some aspects, the securing portions 307, 309 can be configured to allow attachment in an unloaded condition (e.g., attaching the ends together without applying stress) and then having a tightening/shoring feature (e.g., laces that can be tightened) that applies a predictable load or displacement to each portion.

In an aspect, the stiff fabric 310 is also used to create a mounting potion for the m mounting of the securing portions 307, 309 of the sleeve 300. In an aspect, the stiff fabric 310 can form a belt 318 of material for attachment locations. In such aspects, the belt 318 can stretch from one attachment locations (hook and loop toe) to an anchoring location. This belt 318 allows for a rigid hoop of material around the critical bladder location. The elastic fabric 312 is used for two other attachment locations and the remaining anchoring locations on either side of the rigid fabric “belt.” In other aspects, different materials of various stiffness can be utilized to inform order of attachment, attachment placement, and attachment strength. For example, a high elastic connection can be made to approximate the sleeve connectors than rigid connections can be made to solidify the overall assembly.

When the sleeve 300 is placed on the user, the securing portions 307, 309, using a hook and loop system, are placed in the desired locations to achieve the desired sleeve tightness and mechanical attachment, putting the hooks and loops into shear while the underlying fabric is placed into plane tension. The net effect is to achieve “hoop stress” within the stiff fabric sleeve material 310 that places a static load on underlying tissues. The plane tension in the fabric creates a mechanical situation where the bladder 320 held within the sleeve 300 is mechanically affixed against the skin so the pneumatic inflation of the balder translates mechanical energy into the underlying tissues.

To place the correct therapeutic treatment, proper placement of the sleeve 300 on the patient and predictable geometric placement of the bladder 320 within the mechanical cuff is needed. The bladder geometric placement must be maintained in both deflated and inflated conditions. In an aspect, the impermeable bag portion/bladder 320 is configured to transfer therapeutic mechanical energy into the tissue underlying the sleeve. During use, the sleeve 300 locates the bladder 320 in a specific anatomical location on the user and the sleeve 300 enables the bladder 320 to inflate without shifting position or examining away from the underlying tissue intended to be treated. The bladder 320 is sized and positioned within the sleeve 300 to transduce pneumatic pressure into mechanical displacement of the tissue under the sleeve. The amount of pressure within the bladder 320 and the position of the bladder 320 are critical for proper therapy. In an aspect, one bladder 320 is utilized. In other aspects, up to three bladders 320 can be used, providing sequential compression and localized therapy.

In an aspect, the bladder includes tabs or windows of material outside of the bladder area defined by the peripheral weld that enable IPCD cuff assembly, orientation, retention, and inflation. The material outside the peripheral weld is sometimes called the “salvage edge” and is considered the excess material that is typically minimized during design and manufacturing. This material exists is to enable a good peripheral weld and is not loaded during normal use and bladder inflation. The present invention eliminates the need for attachment regions within the bladder inflation area. The present invention reduces the number of welded regions within the bladder inflation area. The present invention avoids adding mechanical loads within the bladder inflation area. The present invention does not impact the inflation geometry of the bladder area.

FIGS. 43-50A-E illustrate embodiments of the bladder with salvage edges retained. As shown in FIG. 43, the bladder 500 includes a bladder inflation region 502 that is defined by the peripheral weld 504. Without changing the design within the bladder inflation region 502, aspects of the invention allow sufficient material in the salvage edge 510 to provide discreet tabs 512 of material that can be used as locations for stitching or other mechanical attachment means. These tabs 512 do not affect the pneumatic integrity of the bladder 500, nor the inflation region 502 of the bladder 500. The tabs 512 provide locations to tack or affix to the sleeve/cuff material, thus enabling mechanical and geometric orientation of the bladder 500 within the cuff (not shown). If excessive load is applied to the bladder 500 within the cuff, the tabs 512 would likely fail prior to the pneumatic area 502 of the bladder 500, thus preserving device function despite the over-load condition. If this overload was applied to a region of material within the bladder's pneumatic area 502, the material failure would risk the function of the device. The shape and size of tabs 512 or “excessive salvage edge material” 510 depends on the design needs, ability to fit the uncut bladder material within the cuff, and the cuff design.

As shown in FIG. 43, tabs 512 are shown attached to a pneumatic bladder design 500. The tabs 512 are made from salvage edge material 510, defined by the peripheral weld 504 and are not contained within the pneumatic region 502 of the bladder 500. As shown, three tabs 512a,b,c are shown as extensions of the bladder material 510 outside of the peripheral weld 504. In other embodiments, various numbers of tabs 512 can extend from any edge of the weld 504. Tear-away zones/perforations 514 (depicted by dotted lines to prevent mechanical forces placed on the tabs 512 from causing failure of the peripheral welds 504 or inflation region 502 of the bladder 500.

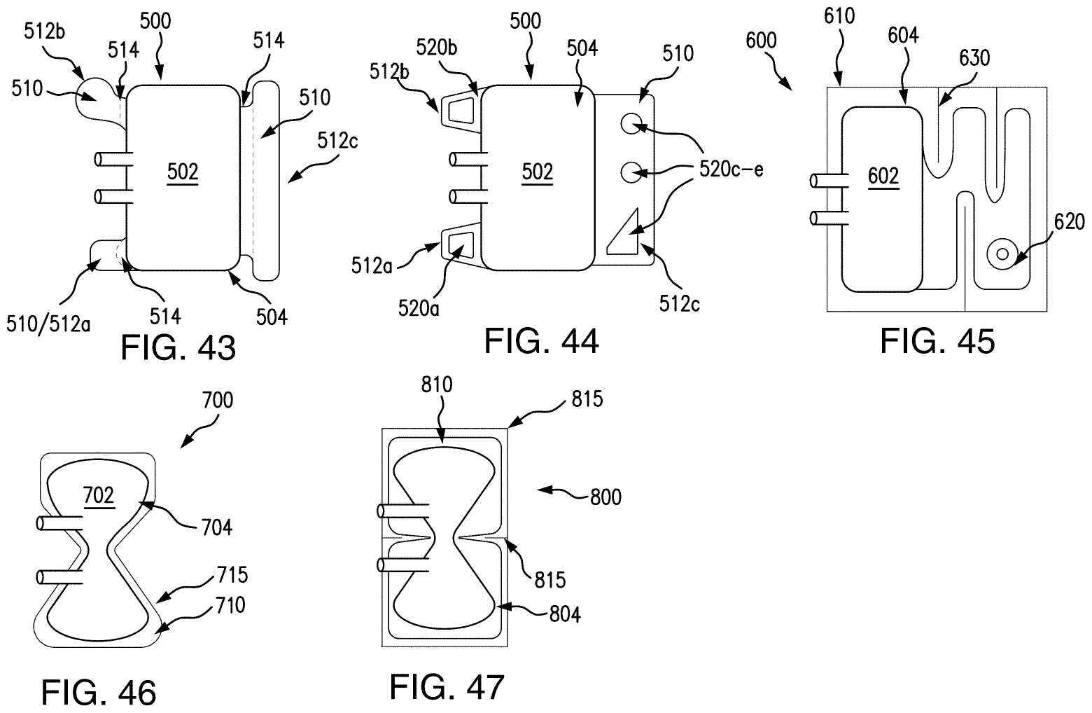

In other aspects, the tabs 512 within the salvage edge 510 can contain windows 520, as shown in FIG. 44. Without the need to change the typical design of bladder inflation region 502, the salvage edge 510 allows sufficient material to provide tabs 512 with windows/apertures 520 that can be used as locations for stitching or other mechanical attachment means. These tabs 512 with windows/apertures 520 do not affect the pneumatic integrity of the bladder 500 nor tis inflation. The tabs 512 with windows 520 provide locations to tack or affix to the sleeve/cuff material through the bladder's salvage edge 510. In such aspects, the edge 510 also enables taking or spot welding of the cuff material within the window 520. Providing windows 520 for mechanical attachments enables mechanical and geometric orientation of the bladder 500 within the cuff. If excessive load is applied to the bladder windows 520, the windows 520 would likely fail prior to transmitting load to the bladder's pneumatic area 502, thus preserving device function despite the over-load condition. For example, a bladder 500 with windows 520 are attached to a sleeve using mechanical connections that pierce the windows 520. If the sleeve is stretched too far and the windows 520 are placed under more mechanical load that the bladder material can withstand, the windows 520 will tear thereby relieving the pneumatic area 502 of the bladder 500 from experiencing the mechanical load that would tear the bladder material in the area of inflation 502, thereby preserving the function of the bladder 500 despite the over-stretched bladder condition. If this overload was applied to a window 520 within the bladder's pneumatic area 502, the material failure would risk the function of the device 20. In various aspects, the windows/apertures 520 in the salvage edge material 510 can be located in nearly any location outside of the pneumatic bladder area 502. The shape of the windows/apertures 520 may be customized to accommodate spot welds on the cuff material around the bladder's periphery 504 or to enable continuous cuff welds 504 along a side or edge of the pneumatic area 502. The number of windows 520 can be at least one or many, depending on the cuff space available and the amount of salvage edge 510 designed into the bladder. Many small windows 520 within the salvage edge material may provide excellent geometric fixation along with redundant mechanical attachments to immure the device against failure.

Referring back to FIG. 44, a bladder 500 with a simple salvage edge 510 according to an aspect of the present invention. A standard bladder 500 is depicted along with tabs 512 containing windows 520. The windows 520 are cut into additional salvage edge material 510 and are not contained within the pneumatic region 502 of the bladder 500. The windows 520 can extend from any edge and be many sizes and shapes. In this image, five windows 520a-f are shown in three tabs 512a-c. The size and location of each window 520 enables formation of various sizes of spot welds or sewing connections with the cuff material. The proximity to the salvage edge 504 can create a desired failure zone that prevents mechanical loads from being transmitted to the pneumatic region 502 of the bladder 500. Tear-away zones or perforations (not shown) could be used in combination with windows 520.

FIG. 45 illustrates a bladder 600 with salvage edge material 610 (tabs or windows or a combination) shaped to form a mechanical interference fit 630 with the cuff weld surrounding the bladder 600. Since the optimal bladder inflation region 602 consists of clean continuous weld lines 604 without deviations or interruptions, a complex salvage edge geometry 610 may be formed to create a complex shape that creates a lock-and-key interference fit 630 with the cuff material. The cuff welds create interference fits with convolutions of the salvage edge. In addition, a window 620 is depicted in the salvage edge 610 along with a spot weld, showing that such features can be combined.

In an aspect, the design of the bladder inflation region 702 can define a shape with invaginations or excursions of the continuous peripheral weld 704 to form a mechanical interference fit with the cuff weld 715 surrounding the bladder 700, as show in FIG. 46. The salvage edge material 710 can also contain tabs and windows or combinations of tabs and windows to enable better cuff attachment to the bladder 700 with complex peripheral weld 704. The benefit of the complex peripheral weld 704 is that the shape of the inflation region 702 can be configured for both optimal therapeutic delivery of energy to the underlying tissues as well as to create a mechanical lock with the welds or attachment points of the cuff material. As shown, no salvage edge 710 is needed to locate or orient the bladder 700 in the cuff. With the modified bladder 700 with invaginations, the cuff weld 715 forms an interference fit that prevents migration, translation, or rotation of the modified bladder 700 in the inflated or deflated state. The cuff welds 715 could also be replaced by line or spot welds and are not required to be continuous (they do not form an air tight seal). Cuff welds 715 can be applied to the modified bladder 700 with at least one line or spot weld within one or both of the bladder invaginations.

Another aspect, as depicted in FIG. 47, includes a bladder 800 with inflation regions 802 with invaginations or excursions of the continuous peripheral weld 804 but maintain the salvage edge material 810 to present a substantially unmodified shape that contains at least one window (not shown) enables mechanical fixation of the bladder within the cuff while preventing rotation, translation, or migration of the bladder 800 relative to the sleeve. The salvage edge material 810 can be used for stitching or other forms of piercing mechanical connections. The salvage edge 810 may contain windows or combinations of tabs and windows to enable better cuff attachment to the bladder with complex peripheral weld. The benefit of the complex peripheral weld 804 is that the shape can be configured for both optimal therapeutic delivery of energy to the underlying tissues as well as to create a mechanical lock with the welds or attachment points of the cuff material. The cuff material welds 815 are also included. In the modified bladder 800 with at least one invagination in the salvage edge material 810, the at least one cuff weld 815 forms an interference fit that prevents migration, translation, or rotation of the modified bladder in the inflated or deflated state. The cuff welds 815 can be replaced by line or spot welds and are not required to be continuous (they do not form an air tight seal). Various combinations of what have been discussed above can be utilized in various aspects of the invention.

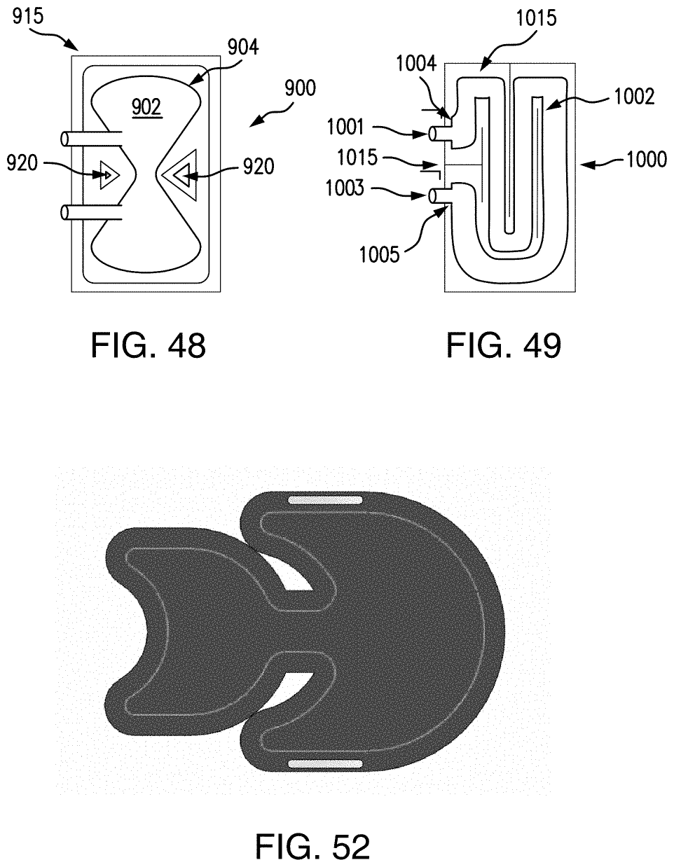

Another aspect, as shown in FIG. 48, includes a bladder 900 with invaginations or excursions of the continuous peripheral weld 904 but maintaining the salvage edge material 910 to present a substantially unmodified shape that may contain at least one window 920 enables mechanical fixation of the bladder 900 within the cuff while preventing rotation, translation, or migration of the bladder 900 relative to the sleeve. The salvage edge material 910 may contain windows 920 to enable better cuff attachment to the bladder 900 with complex peripheral weld 904. The benefit of the complex peripheral weld 904 is that the shape can be configured for both optimal therapeutic delivery of energy to the underlying tissues as well as to create a mechanical lock with the welds or attachment points of the cuff material 915. The benefit of a salvage edge 910 that presents windows 920 for cuff welding 915 is that the unloaded region 910 of the bladder 900 is taking all the mechanical loads for fixation to the cuff. In the modified bladder 900 with at least one window 920 in the salvage edge material 910, the at least one cuff weld 915 forms an interference fit that prevents migration, translation, or rotation of the modified bladder in the inflated or deflated state. The cuff welds 915 are depicted as a spot weld and a small continuous weld that is not required to form an air tight seal (triangle).

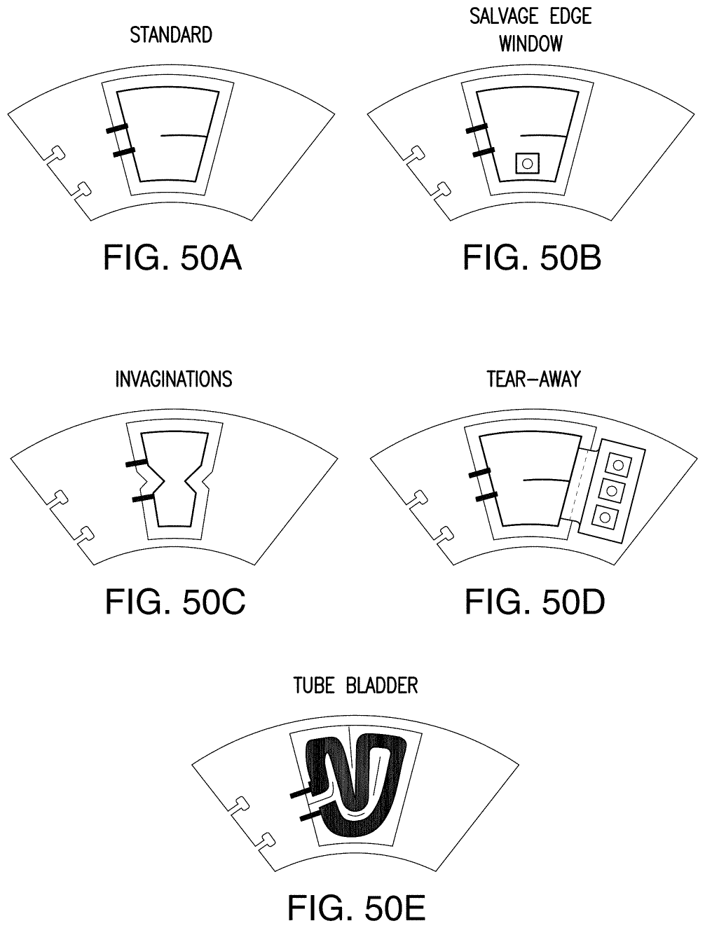

In another aspect, as shown in FIG. 49, the bladder 1000 can include a tube inflation region 1002 to control the inflation shape using cuff welds 1015 that enable mechanical fixation of the bladder 1000 within the cuff while preventing rotation, translation, or migration of the bladder relative to the cuff. The benefit of this design is that it eliminates the peripheral weld and replaces it with two end welds 1004, 1005. In some instances, the welds 1004, 1005 can include a mandrel weld to enable tubing. There is no salvage edge material unless the ends of the tube bladder 1000 are not trimmed and used as the salvage edge material as contemplated above. The shape of the cuff weld 1015 determines the area of inflation 1002. The tube bladder 1000 is depicted with an inlet tube 1001 (mandrel weld) and an exit tube 1003 (another mandrel weld) with no salvage edge. Since the tube has no shape, the cuff welds 1015 create the area and volume of inflation in the assembly. In this embodiment, the inflation height is limited by the tubing diameter if a rigid material is used for the tube bladder (polyethylene terephthlalate, reinforced nylon). If a flexible/elastic tube material is used, the inflation height is controlled by a combination of the cuff weld dimensions, the flexibility of the cuff material, and the elasticity of the tube material (silicone, nitrile rubber, polyisoprene). FIGS. 50A-E illustrate the combination of various shaped bladders within the sleeve, according to various aspects of the present invention.

In another aspect, the inflation is modified by changing the peripheral weld: Most peripheral welds are simple geometries—continuous circles or lines joined by arcuate curves. A non-square, non-circular peripheral weld would form an inflation area that is complex at the periphery, but also more complex in the ability to deliver therapeutic mechanical energy to the underlying tissues. As an example, a circular peripheral weld will result in a circular inflated bladder while a long oval peripheral weld will result in a cigar-shaped inflated bladder. As another example, a peripheral weld with a complex geometry will create various inflated shapes. The shape of the inflated bladder suggests where the mechanical therapeutic energy is being applied to the user. The peripheral weld on the bladder can change to reflect the amount of energy being conveyed into the underlying tissues. FIG. 52 illustrates an example of a complex weld, resulting in a complex shape. Various other shapes and combinations can be used to develop different sized bladders for various therapeutic approaches.

In an aspect, the bladder includes a single bladder device that inflates in segments or sequences of segments. A bladder with a single peripheral weld can defines a single bladder, then placing welds within that single bladder that contain a region where inflation gasses get from a first inflated chamber within the bladder to at least one other inflated chamber within the same bladder defined by the same peripheral weld. The region allowing inflation gasses to pass could be a small gap in the weld that acts to limit gasses traveling into the at least one other inflated chamber so that the net effect is that the at least one other inflated chamber fills with gases after the first inflated chamber.

The small gap in the weld may be a tortuous pathway where resistance to gas flow is controlled by the tortuous aspects of the small gap. The small gap in the weld may be an inclusion of another material that prevents the weld from sealing and where the body of the inclusion material acts as a filter to restrict inflation gas from passing into the at least next inflation chamber. In yet another embodiment, the small gap could include a pressure-relief valve that opens once a critical pressure is reached thereby enabling the first chamber to inflate to a predetermined pressure then allowing the at least next chamber to inflate to another pressure. The small gap in the weld could be controlled by mechanical forces applied during inflation of the first inflation chamber (distended bladder material could open a mechanical valve or material crease that becomes a small gap enabling inflation of an at least next inflation chamber). The small gap in the weld could be controlled by electrical forces sent by an inflation control unit. The electrical forces could open a solenoid valve to enable filling of the at least next inflation chamber and be under digital control. The electrical forces could act on a resistance heater that made the bladder weld material warmer and therefore more pliable thereby enabling opening of the small gap to enable inflation of the at least next chamber. These last two embodiments of the small gap in the weld being controlled by electrical forces could be controlled by the pressure-generating unit or by another signal such as a biofeedback signal demonstrating tissue response to the therapy being delivered (in this embodiment, the pressure generating unit is not controlling the distribution of pressures within the multi-inflation chamber bladder, but other external controls are being applied).

The small gap embodiments could be repeated within a single bladder to create a series of linked chambers. These chambers can be filled in series or in parallel, depending on the mechanisms used to pass inflation gasses from one inflation chamber to the at least next inflation chamber. It may be possible to inflate at least several inflation chambers to different ultimate pressures by including pressure relief valves that vent to the atmosphere (pressure environment outside of the bladder assembly) to prevent those chambers from exceeding the pressure defined by the pressure relief valve. It may be possible to have a series of inflation chambers that inflate to a lower inflation pressure because one of these pressure relief vent valves prevents the ultimate pressure from exceeding the pressure defined in the pressure relief valve. This may enable a device to inflate using a pressure source that has a higher-than-desired value, and metering down the pressure to achieve an ideal therapeutic pressure field that inflates in a predetermined sequence over time to predetermined pressures unique to the pressure chambers within the segmented single bladder. This embodiment of a multi-chambered single bladder with small gap inflation designs could mimic the function of more complicated multi-bladder devices that also require multiple inflation devices or electronic inflation control. This multi-chambered inflation bladder would have the benefit of a single inflation source and basic mechanical (not digital or electrical) controls.

The windows are used for attachment, orientation, retention, and breathing. The function of the windows in the bladder material is for fixing the location of the bladder within the sleeve or cuff. Regardless of placement, these windows orient the bladder before and after inflation. The windows can be placed to help direct mechanical energy during inflation of the bladder. The windows also enable underlying tissues to “breath” around the otherwise impermeable bladder materials.

The device can add windows by the use of continuous salvage edge, a tab on a salvage edge, an untrimmed sheet still attached to the salvage edge, and/or a combination of these features. The window may be a complete through-hole. The window could also be a flap or tab (3-sides of a hole where the 4th side allows the hole to be created when the material is folded over where the uncut edge acts as a living hinge that holds the flap of material onto the window).

In an aspect, the impermeable bag portion 320 can form channels 321 that can be inflated by the modular driving component 100. In an aspect, the channels 321 are oriented in such a fashion that when inflated, the channels 321 apply pressure in a proximal direction; i.e., blood within the limb is pushed towards the heart, The channels 321 apply the pressure against the limb of the subject. In an aspect, the channels 321 of the impermeable bag portion 320 are in fluid communication with each other, as well as the fluid communication pathways 372 that are in communication with the fluid pathway ports 117 of the driving component 100, which are in communication with the pump subsystem 130.

In an aspect, the inflatable sleeve 300 includes a top surface 314 and a bottom surface 316. In an aspect, the top surface 314 hosts the driving component 100. The top surface 314 can host the mounting means 330 configured to releasably retain the driving component 100, as discussed above. In such aspects, the mounting means 330 can be made of the same material as used for the housing 110 of the driving component 100. Further, the mounting means 330 can include securing mechanisms that match those found on the housing 110. The mounting means 330 can include an anchor portion 331 that extends through an opening 301 of the sleeve 300 to establish communication with the air impermeable inflatable bag 320. The fluid communication pathways 372 can extend through the anchor portion 331 to connect the pump subsystem 130 to be in communication with the channels 321 of the inflatable bag 320.

In an aspect, the bottom surface 320 of the inflatable sleeve 300 is configured to engage/come in contact with the skin of the subject. In such instances, it is desirable that that the different securing portions 307, 309 are arranged so that any of the securing means do not come in contact with the skin of the individual. For example, in instances where the securing portions 307, 309 are configured to overlap with one another, the securing means (e.g., hook and loop fasteners) are oriented on the bottom surface 316 of the securing portion 307 and the top surface 314 of the other securing portion 309, preventing the securing means from coming in contact with the skin.

In an aspect, the inflatable sleeve 300 further comprises migration preventing regions that prevent the inflatable sleeve 300 from sliding along the skin of the subject. In an aspect, the migration preventing regions are oriented on the bottom surface 320 of the inflatable sleeve 300. In an aspect, such regions are comprised of textile that has a high skin contact coefficient of friction, but are still comfortable for the subject. In an aspect, the regions can be configured to prevent movement through a combination of their orientation (e.g., perpendicular to the direction of the limb) and configured to cover a certain percentage of the fabric that comes in contact with the skin of the subject. In an aspect, the coverage can be configured to cover approximately 30% or less of the surface area.

In an aspect, the mobile IPC device 20 is configured to run in a comfortable manner for the subject. In such aspects, the mobile IPC device is configured to operate quietly. In such aspects, sound dampening materials, and a low-vibration fluid pump, are used to minimize the perceived sound. Further, the mobile IPC device 20 is more comfortable for the subject to wear because it is more stable and secure compared to currently marketed devices. The IPC device uses components which create a minimal footprint, thereby lowering the device footprint and weight, which in combination of the migration preventing regions, prevents constant re-adjustment of the IPC device 20 on the limb of subjects.

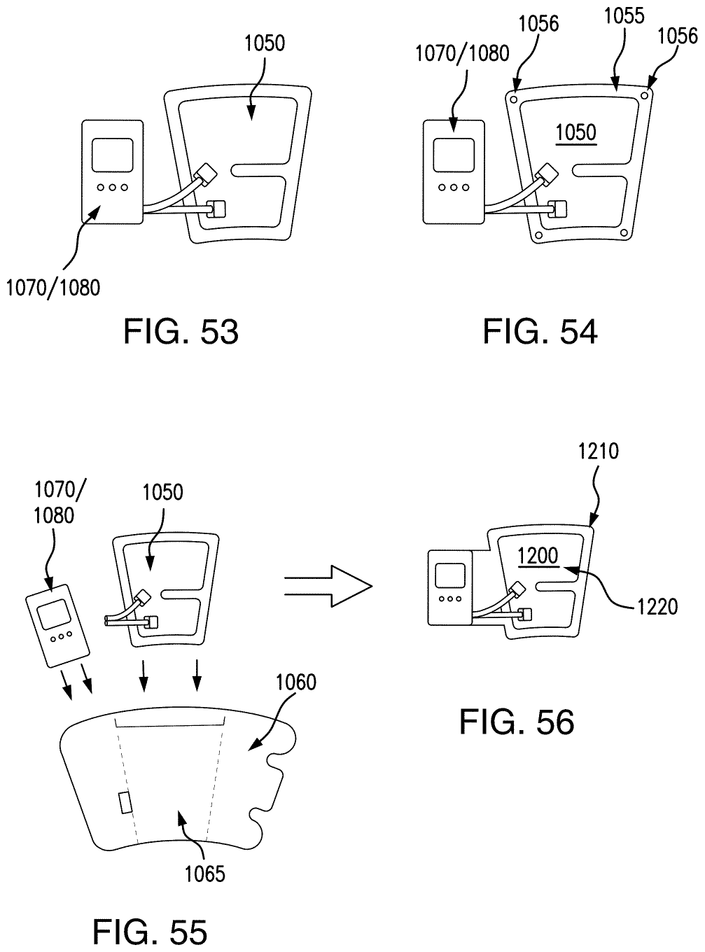

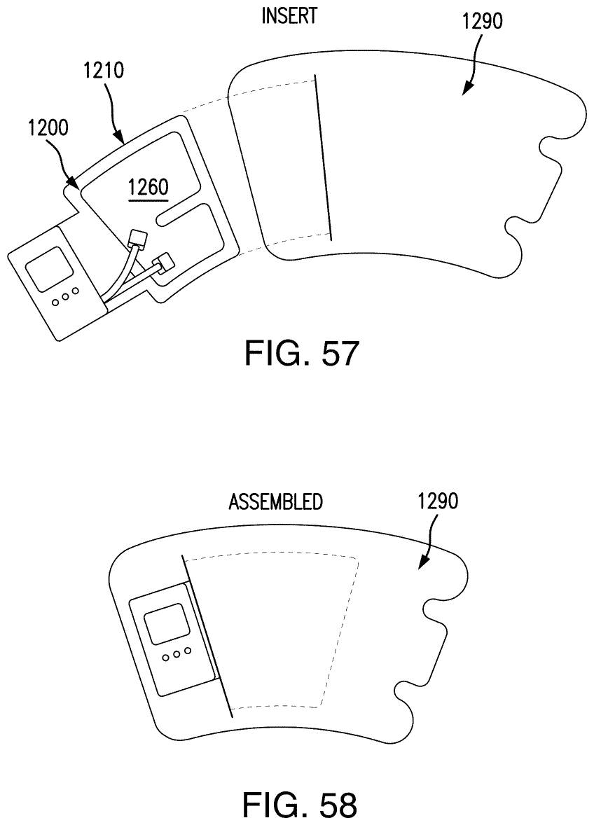

In an aspect, as illustrated in FIGS. 53-58, a bladder 1050 can be configured to be removable from the sleeve 1060, as discussed below. By making the bladder 1050 removable from the sleeve 1060, operation and replacement costs, as well as waste, are reduced. In an aspect, the sleeve contains a feature to capture 1065 the reusable bladder 1050. The sleeve capture feature 1065 can include be a pocket, clip, or snap-on feature. The sleeve capture feature is configured to position the sleeve in close approximation to the tissue being treated. Simultaneously, the sleeve capture feature allows the pump 1070 to be operated and viewed by the user. The sleeve capture feature enables the bladder assembly 1050 to be removed once the user has completed their therapy and the bladder may be applied to another user while the previously attached sleeve is discarded.

In some aspects in which the bladder 1050 is configured to be removed from the sleeve 1060 for additional uses, the bladder 1050 can be configured to be a more integrated component of the driving component 1070, and more specifically the pump 1080, as shown in FIG. 53. In such bladder-pump aspects, the attachment mechanism between the reusable pressure bladder 1050 and the pump 1080 is configured to be more permanent by creating features on the reusable bladder that enable direct permanent or semi-permanent attachment to the pump.

In another aspect, the reusable bladder 1050 is configured to allow easy insertion and removal into the disposable sleeve 1060. The reusable bladder 1050 may contain a stiffening or fixation feature to allow easier insertion into the pocket or sleeve attachment mechanism. For example, the bladder 1050 can include a hard plastic frame 1055 within or around the inflatable bladder 1050, as shown in FIG. 54-55. The stiffening frame 1055 enables the otherwise flexible inflatable bladder 1050 to be inserted into a sleeve capture feature, 1065 like a pocket (see FIG. 55). The frame 1055 may also contain additional fixation features 1056 to attach to the sleeve 1060. Such fixation features 1056 can include like hook-and-loop fasteners or snap features that mate with a similar snap feature on the sleeve. The geometry of the frame 1055 may enable mechanical stability of the assembly after insertion into a pocket within a sleeve where the frame 1055 creates a mechanical attachment by interference or by inserting into sleeve features that ensure the sleeve-bladder assembly has mechanical integrity during the period of use. In another aspect, the frame 1055 may contain tabs that fit into slots within the sleeve 1060 to enable additional mechanical attachment. In another embodiment, the frame 1055 contains flexible or semi-flexible tabs that act as a clip to removably interface with the sleeve.

In another aspect, as shown in FIGS. 56-58, an inflatable bladder 1200 consists of a rigid sheet 1210 of material mated with a flexible or elastic sheet of material 1220 to form a pressure bladder 1200. In this embodiment, the rigid sheet of plastic 1220 acts as a mechanical stiffener to enable easier bladder-insertion into the sleeve 1290 while the flexible or elastic material 1220 enables the bladder 1200 to inflate and expand.

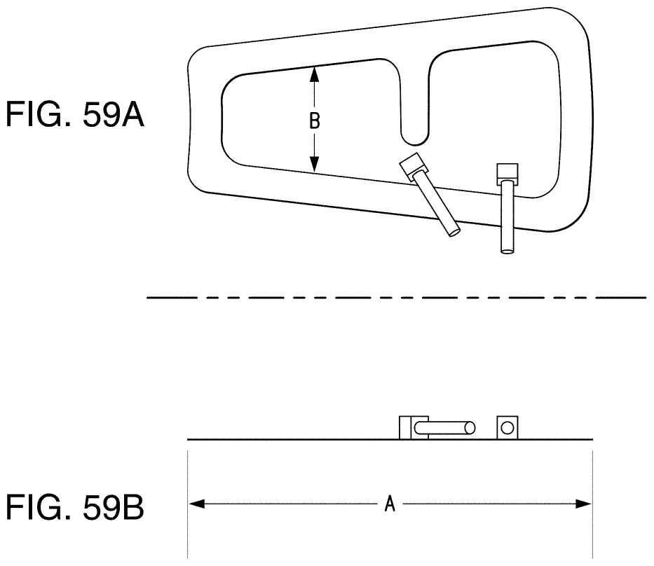

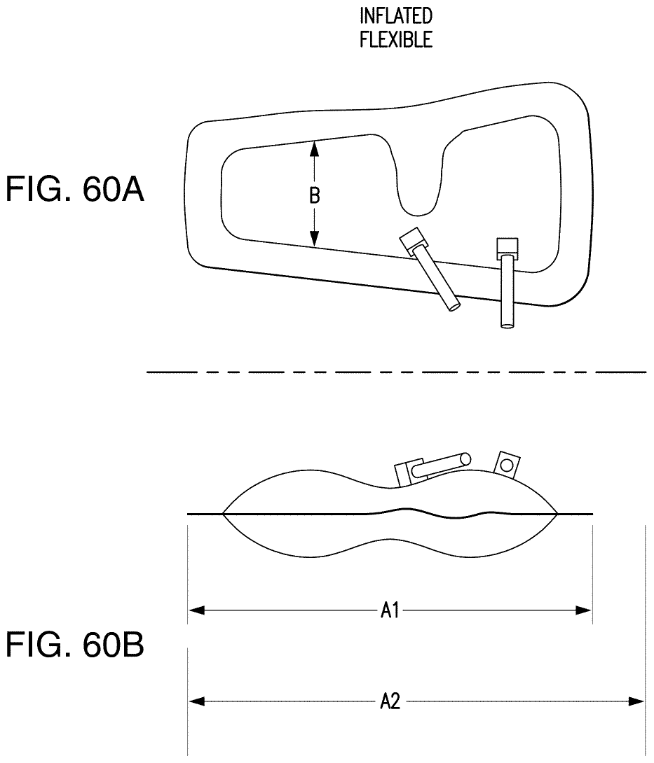

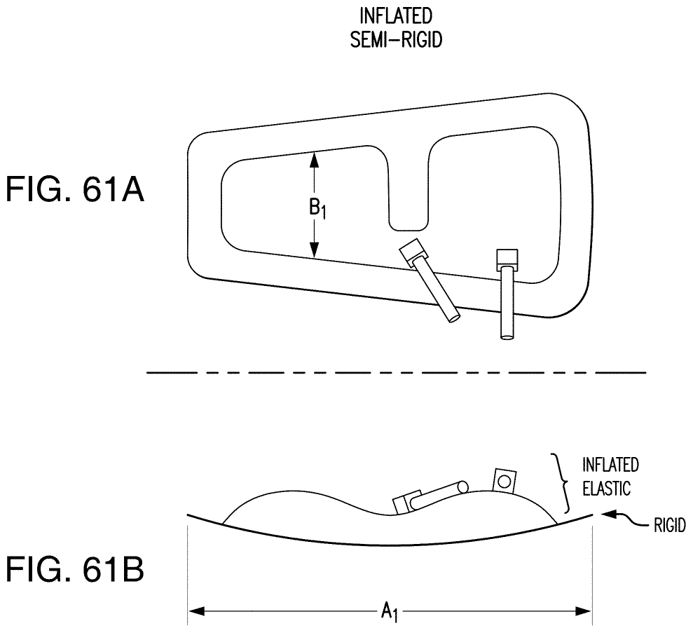

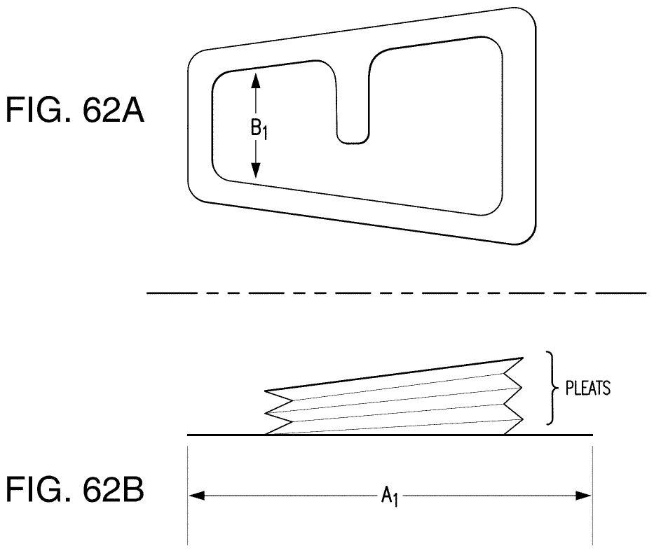

In another aspect, a combination of a rigid material and a flexible material for use with the bladder can add therapeutic benefit as well, as shown in FIGS. 59A-62B. FIGS. 59A-B illustrates a standard bladder having an inflatable region of length B and a bladder length of A. If the top and bottom layers of the bladder are both made of flexible material, the device inflates on both sides, as shown in FIGS. 60A-B. FIGS. 61A-B show a bladder with a top layer of a flexible material and a bottom layer of semi-rigid material. As it is inflated, the semi-rigid material does not deform as much as the flexible layer on top (FIG. 61B). FIGS. 62A-B illustrate a bladder using rigid material, but with a pleated region between the top and bottom. When inflated, the deformity is limited still, but expansion does occur, as shown in FIG. 62B. Using rigid materials resists deformation during inflation and only the flexible or elastic sheet is allowed to expand during inflation. Such a configuration improves the delivery of mechanical energy into the tissue being treated where current designs lose much of the mechanical energy being applied into bladder expansion away from the tissue being treated, thus resulting in lost energy or reduced user therapy.

A configuration where the inflatable bladder 1300 has a rigid face 1310 and a flexible or elastic face improves the therapeutic benefit of IPC devices. Yet another embodiment of an inflatable bladder 1300 using materials that are effectively rigid may involve the use of mechanical bellows on an otherwise entirely rigid bladder. In this embodiment, the amount of bladder inflation can be designed by varying the mechanical stiffness of the pleats forming the bladder. In this embodiment, the amount of bladder inflation can be controlled around the periphery of the bladder by varying the elasticity of the bellows so the ultimate inflated shape of the bladder may be controlled.

In an aspect, the mobile IPC device is configured to comply with several consensus standards. Such standards include, but are not limited to, the following:

ISO 60601-1: Medical electrical equipment; IEC 60601-1-2 Edition 3: 2007-03 Medical electrical equipment—Part 1-2: General requirements for basic safety and essential performance—Collateral standard: Electromagnetic compatibility—Requirements and tests. (EN 60601-1-2); IEC 60601-1-11: 2010—medical electrical equipment—part 1-11: General requirements for basic safety and essential performance—collateral standard: requirements for medical electrical equipment and medical electrical systems used in the home healthcare environment (EN 60601-1-11: 2010); IEC 60601-1-6 2010 3rd edition Medical electrical equipment Part 1-6: General requirements for safety—collateral standard: Usability; IEC 62366: 2007+A1: 2014—Medical Devices—Application of usability engineering to medical devices (EN 62366: 2008); ISO 10993: Biological evaluation of medical devices—FDA expects that a device which contacts intact skin for up to 24 hours will have data to support biocompatibility including: 1. Cytotoxicity (Part 5—Tests for in vitro cytotoxicity), 2. Sensitization (Part 10—Tests for irritation and skin sensitization), 3. Irritation or Intracutaneous Reactivity (Part 10—Tests for irritation and skin sensitization); IEC 62304: Software Lifecycle Processes; ASTM D4169—Standard Practice for Performance Testing of Shipping Containers and Systems; ISO 14971: Risk Management; and ISO 13485: Quality Systems.

Having thus described exemplary embodiments of the invention above, it should be noted by those skilled in the art that the within disclosures are exemplary only and that various other alternatives, adaptations, and modifications may be made within the scope of the present invention. Accordingly, the present invention is not limited to the specific embodiments as illustrated herein.

What is claimed is:

1. A mobile intermittent pneumatic compression (IPC) device for use on a limb of a subject, the device comprising:
a. a driving component comprising;
i. a removable power source;
ii. a pump subsystem;
iii. a computing device configured to control the pump subsystem; and
iv. housing containing the self-contained power source, the pump subsystem, and the computing device; and
b. an inflatable sleeve configured to be placed the limb of the subject, wherein the pump subsystem inflates and deflates the inflatable sleeve, wherein the driving component is removably mounted to the inflatable sleeve.

2. The mobile IPC device from claim 1, wherein driving component is configured to be operable only with the removable power source is connected to the driving component.

3. The mobile IPC device from claim 2, wherein the removable power source is only rechargeable when the removable power source is disconnected from the driving component.

4. The mobile IPC from claim 1, wherein the removable power source is configured to supply power to the driving component for operation of at least 18 hours.

5. The mobile IPC device of claim 1, wherein the inflatable sleeve comprises a sleeve, an inflatable bladder, and a mounting means to mount the driving component to the sleeve.

6. The mobile IPC device of claim 5, wherein the inflatable sleeve further comprises an unique identifying means, wherein the inflatable sleeve is configured to be assigned to a specific patient using the unique identifying means.

7. The mobile IPC device of claim 5, wherein the unique identifying means comprises an RFID chip.

8. The mobile IPC device of claim 5, wherein the sleeve comprises a composite material system.

9. The mobile IPC device of claim 8, wherein the composite material system comprises a stiff fabric and a flexible fabric.

10. The mobile IPC device of claim 5, wherein the inflatable bladder comprises a salvage edge, wherein the salvage edge is configured for securing the inflatable bladder within the sleeve of the inflatable sleeve.

11. The mobile IPC device of claim 5, wherein the inflatable sleeve is configured to be disposable.

12. The mobile IPC device of claim 11, wherein the inflatable bladder is configured to be removable from the sleeve for reuse.

13. A mobile intermittent pneumatic compression (IPC) device for use on a limb of a subject, the device comprising:
a. a driving component comprising;
i. a self-contained removable power source;
ii. a pump subsystem;
iii. a computing device configured to control the pump subsystem;
iv. an inflatable bladder; and
v. housing containing the self-contained power source, the pump subsystem, and the computing device, and the inflatable bladder; and
b. an inflatable sleeve configured to be placed the limb of the subject, wherein the inflatable bladder of the driving component is configured to be inserted into the inflatable sleeve, the inflatable bladder and the pump subsystem configured to inflate and deflate the inflatable, wherein the driving component is removably mounted to the inflatable sleeve.

14. The mobile IPC device of claim 13, wherein the self-contained removable power source is configured to be charged only when not connected to the driving component.

15. The mobile IPC device of claim 13, further comprising a compliance meter that reports user compliance for a current 24-hour period and over a sequence of 24-hour periods.

16. The mobile IPC device of claim 13, wherein the driving component is configured to be inoperable when the self-contained removable power source is removed, and wherein the self-contained removable power source is configured to be recharged only when removed from the driving component.

17. The mobile IPC device of claim 13, wherein the computing device is configured to be reset between use sessions or between users.

18. The mobile IPC device of claim 13, wherein the computing device further comprises a user interface comprising a display, the display configured to show data of the mobile IPC device.

19. The mobile IPC device of claim 13, wherein the computing device is configured to not store any patient-specific data nor is configurable by the patient.

20. The mobile IPC device of claim 13, wherein the computing device is configured to apply a pre-configured pressure cycle.
